(19) United States
(12) Patent Application Publication
(10) Pub. No.: US 2025/0306032 A1
(43) Pub. Date: Oct. 2, 2025

(54) METHODS FOR IDENTIFYING REACTIVE FUNCTIONAL CYSTEINES IN NUCLEAR PROTEINS

(71) Applicant: Talus Bioscience, Inc., Seattle, WA (US)

(72) Inventors: Daniele CANZANI, Seattle, WA (US); William E. FONDRIE, Seattle, WA (US); Lindsay K. PINO, Seattle, WA (US); Alexander FEDERATION, Renton, WA (US)

(21) Appl. No.: 18/865,559

(22) PCT Filed: May 17, 2023
(86) PCT No.: PCT/US2023/067134

§ 371 Date: Nov. 13, 2024

Related U.S. Application Data
(60) Provisional application No. 63/342,908, filed on May 17, 2022.

(51) Int. Cl. G01N 33/68; G01N 1/34

(52) U.S. Cl. CPC G01N 33/6875; G01N 1/34; G01N 33/6848

(57) ABSTRACT

The disclosure relates to compositions and methods for identifying reactive cysteines on nuclear proteins from cell samples. Further wherein the method includes contacting live cells, dead cells, cell lysates, nuclei, or a nuclear fraction obtained from cells with an electrophilic probe, and performing mass spectrometry analysis of proteins present in the cytoplasmic fraction.

### CROSS-REFERENCE TO RELATED APPLICATIONS

This application claims the benefit of and priority to U.S. Provisional Patent Application No. 63/342,908, filed on May 17, 2022, the disclosure of which is incorporated herein by reference in its entirety for all purposes.

### STATEMENT REGARDING SEQUENCE LISTING

The Sequence Listing XML associated with this application is provided in XML file format and is hereby incorporated by reference into the specification. The name of the XML file containing the Sequence Listing XML is TLUS_004_01WO_ST26.xml. The XML file is 3,695 bytes, and created on May 16, 2023, and is being submitted electronically via USPTO Patent Center.

### TECHNICAL FIELD

The disclosure relates to compositions and methods for identifying reactive functional cysteines on nuclear proteins from live cells.

### BACKGROUND

Gene expression is tightly regulated by interactions between protein transcription factors and DNA. Abnormal transcription factor activity has been identified as a driver of various cancers, so transcription factors are desirable candidate targets for drug development. However, most transcription factors have been considered “undruggable” because they lack the well-defined protein structural elements that pair with traditional drug design. Unconventional covalent drugs that react with protein cysteine residues offer promise for targeting transcription factors, but comprehensive mapping of reactive cysteines that impact transcription factor function has not been achieved to date.

The present disclosure meets this need by providing methods to identify functional reactive cysteines, including those present in nuclear proteins obtained from cancer cells.

### BRIEF DESCRIPTION OF THE DRAWINGS

FIG. 1 shows that cells treated with an electrophilic probe, such as n-methyl iodoacetamide (NM-IAA), can be harvested, and then fractionated to isolate the nuclear proteome. Nuclear extracts are highly enriched with transcription factors and other chromatin-associated proteins.

FIG. 2A shows mass spectrum of peptide YHDPNFVPAAFVCSK (SEQ ID NO: 1) from the transcription factor CCCTC-binding factor (CTCF) in THP-1 cells. The mass spectrum shows that the cysteine in the peptide was modified by NM-IAA (bottom) compared to the control sample (top), which was alkylated with iodoacetamide (IAA) before digestion with trypsin. The mass shift associated with NM-IAA is 71.03 Dalton (Da). FIG. 2B shows that electrophilic molecules other than NM-IAA can be used to identify reactive cystines. THP-1 cells were treated with 10 μM biotin polyethyleneoxide iodoacetamide (Biotin-IA). Biotin-IA reacted with a cysteine in the peptide SDAYYCTGDVTAWTK (SEQ ID NO: 2) from the poly(ADP-ribose) polymerase-1 (PARP1) protein.

FIG. 3 shows the number of transcription factor cysteines that were modified by NM-IAA in THP-1 monocytic leukemia cells across three replicates. The high overlap indicates the reproducibility of the chromatin enriching salt separation (ChESS) approach for identifying reactive cysteines.

FIG. 4 shows that seven cancer cell lines (THP-1, SK-N-AS, Kelly, RD, RH4, RH30, and IMR32) were treated with NM-IAA. Nuclear and chromatin proteins were isolated from each sample using ChESS and analyzed by mass spectrometry. 4245 cysteines were found to be modified across the nuclear proteome.

FIG. 5 shows that chromatin protein extraction facilitates the identification of transcription factors. Of 576 transcription factors that were identified, a high proportion of them were found to have cysteines that react with NM-IAA.

FIG. 6 shows that NM-IAA or other electrophilic probes can be applied directly to isolated nuclei instead of live cells to identify transcription factors with reactive cysteines.

FIG. 7A shows that an EZH2 cysteine was found to be modified by an electrophilic molecule, T001-1540, that contains a propynamide reactive site. FIG. 7B shows that EZH2 was significantly depleted by T001-1540 treatment.

### SUMMARY OF THE INVENTION

The disclosure provides methods for identifying reactive cysteine residues that combine a novel electrophilic probe and nuclear protein enrichment to enhance reactive transcription factor cysteine identification for drug discovery.

In one aspect, the disclosure provides a method for identifying reactive cysteine residues present in cellular proteins, the method comprising:
(a) contacting cells, optionally live cells, with an electrophilic probe, e.g., n-methyl iodoacetamide (NM-IAA);
(b) harvesting and lysing the cells following (a);
(c) separating nuclei from the lysed cells, and optionally collecting the supernatant from the separated nuclei as a cytoplasmic fraction;
(d) extracting nuclear proteins from the separated nuclei by resuspending the separated nuclei of (c) in a buffer comprising salt (optionally wherein the buffer does not comprise polycations);
(e) separating insoluble chromatin after (d), and optionally collecting the supernatant from the separated insoluble chromatin as a nuclear fraction; and
(f) performing mass spectrometry analysis of proteins present in the cytoplasmic fraction and/or nuclear fraction to identify reactive cysteines modified by the electrophilic probe, e.g., NM-IAA,
   thereby identifying reactive cysteines present in proteins in the cells and/or how cysteine engagement by an electrophilic probe may change protein localization, optionally wherein the proteins are nuclear proteins, e.g., transcription factors.

In another aspect, the disclosure provides a method for identifying reactive cysteine residues present in cellular proteins, the method comprising:
(a) contacting dead cells or cell lysates with an electrophilic probe, e.g., n-methyl iodoacetamide (NM-IAA);
(b) optionally lysing the dead cells, and separating nuclei from the lysed cells or cell lysate, and optionally collecting the supernatant from the separated nuclei as a cytoplasmic fraction;
(c) extracting nuclear proteins from the separated nuclei by resuspending the separated nuclei of (b) in a buffer comprising salt (optionally wherein the buffer does not comprise polycations);
(d) separating insoluble chromatin after (c), and optionally collecting the supernatant from the separated insoluble chromatin as a nuclear fraction; and
(e) performing mass spectrometry analysis of proteins present in the cytoplasmic fraction and/or nuclear fraction to identify reactive cysteines modified by the electrophilic probe, e.g., NM-IAA,
   thereby identifying reactive cysteines present in proteins in the cells and/or how cysteine engagement by an electrophilic probe may change protein localization, optionally wherein the proteins are nuclear proteins, e.g., transcription factors.

In another aspect, the disclosure provides a method for identifying reactive cysteine residues present in cellular proteins, the method comprising:
(a) contacting nuclei obtained from cells with an electrophilic probe, e.g., n-methyl iodoacetamide (NM-IAA);
(b) extracting nuclear proteins from the separated nuclei by resuspending the separated nuclei of (a) in a buffer comprising salt (optionally wherein the buffer does not comprise polycations);
(c) separating insoluble chromatin after (b), and optionally collecting the supernatant from the separated insoluble chromatin as a nuclear fraction; and
(d) performing mass spectrometry analysis of proteins present in the cytoplasmic fraction and/or nuclear fraction to identify reactive cysteines modified by the electrophilic probe, e.g., NM-IAA,
   thereby identifying reactive cysteines present in proteins in the cells and/or how cysteine engagement by an electrophilic probe may change protein localization, optionally wherein the proteins are nuclear proteins, e.g., transcription factors.

In one aspect, the disclosure provides a method for identifying reactive cysteine residues present in cellular proteins, the method comprising:
(a) contacting a nuclear fraction obtained from cells with an electrophilic probe, e.g., n-methyl iodoacetamide (NM-IAA); and
(b) performing mass spectrometry analysis of proteins present in the cytoplasmic fraction and/or nuclear fraction to identify reactive cysteines modified by the electrophilic probe, e.g., NM-IAA,
   thereby identifying reactive cysteines present in proteins in the cells, optionally wherein the proteins are nuclear proteins, e.g., transcription factors.

In another aspect, the disclosure provides a method for identifying reactive cysteine residues present in cellular proteins, the method comprising:
(a) contacting cells, optionally live cells, with an electrophilic probe, e.g., n-methyl iodoacetamide (NM-IAA);
(b) harvesting and lysing the cells following (a);
(c) separating nuclei from the lysed cells, and optionally collecting the supernatant from the separated nuclei, wherein the supernatant is enriched for cytosolic proteins;
(d) extracting nucleoplasm associated proteins, euchromatin associated proteins, and/or heterochromatin associated proteins from the separated nuclei as follows:
   (i) resuspending the nuclei of (c) in an isotonic buffer comprising salt (optionally wherein the buffer does not comprise polycations) to extract nucleoplasm associated proteins, and then separating insoluble chromatin and collecting supernatant, wherein the supernatant is enriched for nucleoplasm associated proteins;
   (ii) resuspending the insoluble chromatin of (i) in a low salt buffer (optionally wherein the buffer does not comprise polycations), wherein the low salt buffer comprises an increased salt concentration as compared to the isotonic buffer, to extract euchromatin associated proteins, and then separating insoluble chromatin and collecting supernatant, wherein the supernatant is enriched for euchromatin associated proteins; and/or
   (iii) resuspending the insoluble chromatin of (ii) in a high salt buffer (optionally wherein the buffer does not comprise polycations), wherein the high salt buffer comprises an increased salt concentration as compared to the low salt buffer, to extract heterochromatin associated proteins, and then separating insoluble chromatin and collecting supernatant, wherein the supernatant is enriched for heterochromatin associated proteins;
thereby isolating supernatant enriched in cytoplasmic proteins, nucleoplasm associated proteins, euchromatin associated proteins, and/or heterochromatin associated proteins from the cells; and
(e) performing mass spectrometry analysis of proteins present in one or more of the collected supernatants to identify reactive cysteines modified by the electrophilic probe, e.g., NM-IAA, thereby identifying reactive cysteines present in proteins in the cells, optionally wherein the proteins are nuclear proteins, e.g., transcription factors.

In another aspect, the disclosure provides a method for identifying reactive cysteine residues present in cellular proteins, the method comprising:
(a) contacting dead cells or cell lysate with an electrophilic probe, e.g., n-methyl iodoacetamide (NM-IAA);
(b) optionally lysing the dead cells, and separating nuclei from the lysed cells or cell lysate, and optionally collecting the supernatant from the separated nuclei as a cytoplasmic fraction;
(c) separating nuclei from the lysed cells, and optionally collecting the supernatant from the separated nuclei, wherein the supernatant is enriched for cytosolic proteins;
(d) extracting nucleoplasm associated proteins, euchromatin associated proteins, and/or heterochromatin associated proteins from the separated nuclei as follows:
   (i) resuspending the nuclei of (c) in an isotonic buffer comprising salt (optionally wherein the buffer does not comprise polycations) to extract nucleoplasm associated proteins, and then separating insoluble chromatin and collecting supernatant, wherein the supernatant is enriched for nucleoplasm associated proteins;
   (ii) resuspending the insoluble chromatin of (i) in a low salt buffer (optionally wherein the buffer does not comprise polycations), wherein the low salt buffer comprises an increased salt concentration as compared to the isotonic buffer, to extract euchromatin associated proteins, and then separating insoluble chromatin and collecting supernatant, wherein the supernatant is enriched for euchromatin associated proteins; and/or
   (iii) resuspending the insoluble chromatin of (ii) in a high salt buffer (optionally wherein the buffer does not comprise polycations), wherein the high salt buffer comprises an increased salt concentration as compared to the low salt buffer, to extract heterochromatin associated proteins, and then separating insoluble chromatin and collecting supernatant, wherein the supernatant is enriched for heterochromatin associated proteins;
thereby isolating supernatant enriched in cytoplasmic proteins, nucleoplasm associated proteins, euchromatin associated proteins, and/or heterochromatin associated proteins from the cells; and
(e) performing mass spectrometry analysis of proteins present in one or more of the collected supernatants to identify reactive cysteines modified by the electrophilic probe, e.g., NM-IAA,
   thereby identifying reactive cysteines present in proteins in the cells, optionally wherein the proteins are nuclear proteins, e.g., transcription factors.

In another aspect, the disclosure provides a method for identifying reactive cysteine residues present in cellular proteins, the method comprising:
(a) contacting nuclei obtained from cells with an electrophilic probe, e.g., n-methyl iodoacetamide (NM-IAA);
(b) extracting nucleoplasm associated proteins, euchromatin associated proteins, and/or heterochromatin associated proteins from the separated nuclei as follows:
   (i) resuspending the nuclei of (a) in an isotonic buffer comprising salt (optionally wherein the buffer does not comprise polycations) to extract nucleoplasm associated proteins, and then separating insoluble chromatin and collecting supernatant, wherein the supernatant is enriched for nucleoplasm associated proteins;
   (ii) resuspending the insoluble chromatin of (i) in a low salt buffer (optionally wherein the buffer does not comprise polycations), wherein the low salt buffer comprises an increased salt concentration as compared to the isotonic buffer, to extract euchromatin associated proteins, and then separating insoluble chromatin and collecting supernatant, wherein the supernatant is enriched for euchromatin associated proteins; and/or
   (iii) resuspending the insoluble chromatin of (ii) in a high salt buffer (optionally wherein the buffer does not comprise polycations), wherein the high salt buffer comprises an increased salt concentration as compared to the low salt buffer, to extract heterochromatin associated proteins, and then separating insoluble chromatin and collecting supernatant, wherein the supernatant is enriched for heterochromatin associated proteins;
thereby isolating supernatant enriched in cytoplasmic proteins, nucleoplasm associated proteins, euchromatin associated proteins, and/or heterochromatin associated proteins from the cells; and
(c) performing mass spectrometry analysis of proteins present in one or more of the collected supernatants to identify reactive cysteines modified by the electrophilic probe, e.g., NM-IAA,
   thereby identifying reactive cysteines present in proteins in the cells, optionally wherein the proteins are nuclear proteins, e.g., transcription factors.

In another aspect, the disclosure provides a method for identifying reactive cysteine residues present in cellular proteins, the method comprising:
(a) contacting one or more nuclear fraction obtained from cells with an electrophilic probe, e.g., n-methyl iodoacetamide (NM-IAA), wherein the one or more nuclear fraction is optionally enriched for nucleoplasm associated proteins, euchromatin associated proteins, and/or heterochromatin associated proteins, optionally where the nuclear fraction was obtained from nuclei as follows:
   (i) resuspending the nuclei of (a) in an isotonic buffer comprising salt (optionally wherein the buffer does not comprise polycations) to extract nucleoplasm associated proteins, and then separating insoluble chromatin and collecting supernatant, wherein the supernatant is enriched for nucleoplasm associated proteins;
   (ii) resuspending the insoluble chromatin of (i) in a low salt buffer (optionally wherein the buffer does not comprise polycations), wherein the low salt buffer comprises an increased salt concentration as compared to the isotonic buffer, to extract euchromatin associated proteins, and then separating insoluble chromatin and collecting supernatant, wherein the supernatant is enriched for euchromatin associated proteins; and/or
   (iii) resuspending the insoluble chromatin of (ii) in a high salt buffer (optionally wherein the buffer does not comprise polycations), wherein the high salt buffer comprises an increased salt concentration as compared to the low salt buffer, to extract heterochromatin associated proteins, and then separating insoluble chromatin and collecting supernatant, wherein the supernatant is enriched for heterochromatin associated proteins;
thereby isolating supernatant fractions enriched in cytoplasmic proteins, nucleoplasm associated proteins, euchromatin associated proteins, and/or heterochromatin associated proteins from the cells; and
(b) performing mass spectrometry analysis of proteins present in one or more of the collected supernatants to identify reactive cysteines modified by the electrophilic probe, e.g., NM-IAA,
   thereby identifying reactive cysteines present in proteins in the cells, optionally wherein the proteins are nuclear proteins, e.g., transcription factors.

In particular embodiments of any of the methods disclosed herein, the methods further comprise homogenizing, washing, and/or pelleting the cells before lysing the cells. In certain embodiments, the cells are lysed by contacting them with a detergent, optionally wherein the detergent solution comprises NP40 detergent solution, and optionally wherein the NP40 is present at a concentration of from about 0.1% to about 4%.

In particular embodiments of any of the methods disclosed herein, the methods further comprise incubating the nuclei and/or insoluble chromatin in the buffer(s) before separating the nuclei or insoluble chromatin from the supernatant. In certain embodiments, the nuclei are incubated at a temperature of about 4° C. In certain embodiments, the nuclei are incubated for a period of about 30 minutes. In certain embodiments, the salt comprises sodium chloride (NaCl).

In particular embodiments of any of the methods disclosed herein, the methods further comprise quenching the nuclei, optionally after separating the nuclei from the lysed cells. In certain embodiments, the nuclei are quenched with ethylenediaminetetraacetic acid (EDTA). In certain embodiments, the EDTA is present at a concentration of from 0.1 mM to 10 mM.

In particular embodiments of any of the methods disclosed herein, the methods further comprise treating the nuclei with a nuclease. In certain embodiments, the nuclei are treated with the nuclease at a temperature of about 37° C. and/or for a period of about 5 minutes.

In particular embodiments of any of the methods disclosed herein, the methods further comprise adding a surfactant to one or more supernatant and/or insoluble chromatin.

In another aspect, the disclosure provides methods for determining if a condition alters the subcellular location of one or more cellular protein, comprising:
(a) performing any of the methods disclosed herein on cells subjected to a first condition (or cell lysates, nuclei and/or nuclear fractions obtained therefrom);
(b) performing any of the methods disclosed herein on cells subjected to a second condition (or cell lysates, nuclei and/or nuclear fractions obtained therefrom); and
(c) determining the subcellular location of one or more cellular protein following step (a) and step (b), optionally wherein the subcellular location is selected from the group consisting of cytoplasmic, nucleoplasm associated, euchromatin associated, and heterochromatin associated,
   thereby determining whether the first or second condition alters the subcellular location of the one or more cellular proteins. In certain embodiments, the proteins are transcription factors.

In certain embodiments, the cells subjected to the first and second conditions are live cells.

In certain embodiments, the first and second conditions are selected from:
(a) a first and second environmental condition;
(b) a first and second cell state;
(c) before and after treatment with one or more candidate therapeutic agent;
(d) before and after treatment with a small molecule degradation compound;
(e) before and after a gene modification of the cells, optionally by genome editing;
(f) before and after expression of a transgene in the cells;
(g) a first and second thermal condition; and
(h) healthy cells versus diseased or injured cells.

### DETAILED DESCRIPTION

The disclosure provides compositions and methods to identify reactive cysteines, including those present in nuclear proteins, such as transcription factors, obtained from cells. In certain embodiments, the methods combine use of an electrophilic probe and nuclear protein enrichment to enhance reactive cysteine identification. In particular embodiments, the electrophilic probe is n-methyl iodoacetamide (NM-IAA), which is highly reactive towards cysteine residues. When NM-IAA is given to live cells, it enters the cell and may react with cysteines that are exposed. The NM-IAA forms a covalent bond with the cysteine, and the resulting change in mass to the cysteine can be identified, e.g., using a mass spectrometry approach.

In certain embodiments, to enhance the identification of reactive cysteines, the NM-IAA molecule is given to cells which are then fractionated using chromatin enriching salt separation (ChESS). ChESS uses a series of sequential buffer washes of increasing ionic strength to separate the cellular proteome into one or more of cytosolic, nucleoplasm, euchromatin, and heterochromatin fractions. In particular embodiments, ChESS utilizes two or more, three or more, or four or more sequential buffer washes, and in particular embodiments, separates the cellular proteome into two or more, three or more, or all four of cytosolic, nucleoplasm, euchromatin, and heterochromatin fractions. Those fractions separate proteins into different samples which are then individually acquired on a mass spectrometer. Separating the proteins in this manner allows the mass spectrometer to identify more reactive cysteines based on the NM-IAA mass modification. The nucleoplasm, euchromatin, and heterochromatin fractions from the ChESS experiment are of importance, because they contain transcription factors. In particular, the euchromatin and heterochromatin fractions provided by ChESS enrich for transcription factors and other gene regulatory proteins, which facilitates the detection of reactive cysteines on transcription factors, including those with clinical relevance. ChESS methods include but are not limited to those disclosed in U.S. Patent Application Publication No. 20200033358, which is incorporated herein by reference in its entirety.

The cysteines that have been modified with NM-IAA and are identified by mass spectrometry can used to guide therapeutic drug design activities. Cellular fractionation provides enhanced detection of NMIAA-modified peptides as compared to the analysis of whole-cell proteomes. Furthermore, the ability to localize transcription factors to a cellular or nuclear fraction provides insights into the functional consequences of an individual reactive cysteine, such as perturbations to chromatin binding. Profiling NM-IAA reactivity in various cancer cell lines also uncovers unique reactive transcription factors, which may offer new avenues for drug development.

The disclosed methods allow for facile labeling of reactive cysteines throughout the proteome and enhance identification of reactive cysteines on transcription factors. The disclosed methods are advantageous, because they only require a single-step reaction that doesn't require chemical enrichment, and they can be used to separate the proteins of the cell into different fractions, which enhances the identification of transcription factors by mass spectrometry. Proteome fractionation can also increase the number of identifiable reactive cysteines with mass spectrometry. The fractionation of the cell nuclear proteins allows the disclosed methods a greater ability to detect reactive cysteines on transcription factors. The method is also compatible with live cells, whereas previous methods treat proteins in cell lysate.

Overall, the use of NM-IAA with ChESS provides a facile and reliable method to profile reactive cysteines of transcription factors and other cellular proteins. ChESS fractionation may also be coupled to standard affinity-based protein profiling methods for identification and quantitation of reactive cysteines, especially for nuclear proteins.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an” and “the” include plural referents unless the context clearly dictates otherwise.

In this application, the use of “or” means “and/or” unless stated otherwise. Furthermore, use of the term “including” as well as other forms, such as “include,” “includes,” and “included,” is not limiting.

As used herein, ranges and amounts may be expressed as “about” a particular value or range. About also includes the exact amount. Hence “about 5 uL” means “about 5 uL” and also “5 uL.” Generally, the term “about” includes an amount that would be expected to be within experimental error. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Throughout this specification, unless the context requires otherwise, the word “comprise”, or variations such as “comprises” or “comprising”, will be understood to imply the inclusion of a stated element, integer, or group of elements or integers but not the exclusion of any other element, integer, or group of elements or integers.

“Decrease” or “inhibit” may refer to a decrease or inhibition of at least 5%, for example, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%, for example, as compared to a reference or control level. Decrease or inhibit also means decreases or inhibition of at least 1-fold, for example, at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 500, at least 1000-fold or more, for example, as compared to the level of a reference or the level in control cells or tissue.

“Increase” may refer to an increase of at least 5%, for example, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100%, for example, as compared to a reference level or the level in control cells or tissue. Increase also means increases of at least 1-fold, for example, at least 1, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 500, at least 1000-fold or more, for example, as compared to the level of a reference or the level in control cells or tissue.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

### Methods

The methods disclosed herein are used to identify reactive cysteines present in cellular proteins. Generally, the methods include contacting cells (also referred to as a cell sample) or components thereof with an electrophilic probe that binds to reactive cysteines present in cellular proteins. In particular embodiments, the cells are live cells, and in particular embodiments, the cellular proteins are cytoplasmic and/or nuclear proteins. In other embodiments, the methods comprise contacting nuclei, dead cells, cell lysates, or fractions of nuclear proteins, such as ChESS fractions. Once the electrophilic probe has bound to reactive cysteines within the cellular proteins, the cells are harvested, and proteins are extracted and analyzed by mass spectrometry to identify proteins, or fragments thereof, that are labeled or modified by the electrophilic probe.

### Cell Samples

Cell samples include, but are not limited to, live cells, dead cells, lysed cells, nuclei, cytoplasmic cellular fractions, and nuclear cellular fractions, such as nucleoplasm, euchromatin, and heterochromatin fractions.

The cells may be from a variety of sources. For examples, the cells may be cell lines, including primary cell lines, or they may be obtained from a tissue, organ, or organism, e.g., mammalian cells. Cells may also be dead cells. Cells may also be present in cultured organoids, e.g., in vitro organoids produced from cells obtained from a cell, tissue, organ, or organism, e.g., a mammal. In particular embodiments, the cells are mammalian cells or are obtained from a mammal. In particular embodiments, the mammalian cell is an epithelial cell, a connective tissue cell, a hormone secreting cell, a nerve cell, a skeletal muscle cell, a blood cell, an immune system cell, or a stem cell. In certain embodiments, the cells are obtained from blood, urine, stool, saliva, lymph fluid, cerebrospinal fluid, synovial fluid, cystic fluid, ascites, pleural effusion, amniotic fluid, chorionic villus sample, vaginal fluid, interstitial fluid, nasal swab sample, buccal swab sample, sputum, bronchial lavage, Pap smear sample, or ocular fluid. The cell sample may comprise cells obtained from a blood sample, an aspirate sample, or a smear sample. Cells may be obtained from a biopsy sample.

In certain embodiments, the cells are derived from a cell line. Illustrative cell lines include, but are not limited to, 293 cells, Jurkat cells, CHO cells, Hela cells, CV-1 cells, 293A cell line, 293 FT cell line, 293F cell line, 293 H cell line, HEK 293 cell line, CHO DG44 cell line, CHO—S cell line, CHO-K1 cell line, Expi293F™ cell line, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cell line, FreeStyle™ CHO-S cell line, GripTite™ 293 MSR cell line, GS-CHO cell line, HepaRG™ cell line, T-REx™ Jurkat cell line, Per.C6 cell line, T-REX™-293 cell line, T-REX™-CHO cell line, T-REx™-HeLa cell line, NC-HIMT cell line, and PC12 cell line.

The cells may comprise healthy and/or diseased or damaged cells. For example, in certain embodiments, the cells are obtained from a mammal diagnosed with a disease or disorder, such as, e.g., a cancer or tumor, an inflammatory disease or disorder, an immune disease or disorder, a genetic disease or disorder, a metabolic disease or disorder, a cardiac disease or disorder, ischemia or reperfusion injury, or an infection, e.g., infection by bacteria, virus, fungi, etc. Cells may be obtained from a healthy or a diseased tissue or organ.

In certain embodiments, the cells are cancerous cells. The cancerous cells may from a cancer which may be a solid tumor or a hematologic malignancy. The cancerous cell sample may comprise cells obtained from a solid tumor. The solid tumor may include a sarcoma or a carcinoma. Exemplary sarcoma cell sample may include, but are not limited to, cell sample obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (WE), malignant fibrous histiocytoma (WE) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Illustrative carcinoma cell samples may include, but are not limited to, cell samples obtained from an anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

The cancerous cell sample may comprise cells obtained from a hematologic malignancy. Hematologic malignancy may comprise a leukemia, a lymphoma, a myeloma, a non-Hodgkin's lymphoma, or a Hodgkin's lymphoma. The hematologic malignancy may be a T-cell based hematologic malignancy. The hematologic malignancy may be a B-cell based hematologic malignancy. Exemplary B-cell based hematologic malignancy may include, but are not limited to, chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy may include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

The cell sample may comprise circulating tumor cells. A circulating tumor cell sample may comprise lymphoma cells, fetal cells, apoptotic cells, epithelia cells, endothelial cells, stem cells, progenitor cells, mesenchymal cells, osteoblast cells, osteocytes, hematopoietic stem cells, foam cells, adipose cells, transcervical cells, circulating cardiocytes, circulating fibrocytes, circulating cancer stem cells, circulating myocytes, circulating cells from a kidney, circulating cells from a gastrointestinal tract, circulating cells from a lung, circulating cells from reproductive organs, circulating cells from a central nervous system, circulating hepatic cells, circulating cells from a spleen, circulating cells from a thymus, circulating cells from a thyroid, circulating cells from an endocrine gland, circulating cells from a parathyroid, circulating cells from a pituitary, circulating cells from an adrenal gland, circulating cells from islets of Langerhans, circulating cells from a pancreas, circulating cells from a hypothalamus, circulating cells from prostate tissues, circulating cells from breast tissues, circulating cells from circulating retinal cells, circulating ophthalmic cells, circulating auditory cells, circulating epidermal cells, circulating cells from the urinary tract, or combinations thereof.

In certain embodiments, the cells are immune cells. The immune cells may be naturally derived or engineered, for example, to express an exogenous receptor. Immune cells develop from stem cells in the bone marrow and become different types of mature cells. Exemplary immune cells may include, but are not limited to, lymphocytes (e.g., T cells, B cells), neutrophils, eosinophils, basophils, mast cells, monocytes, macrophages, dendritic cells, and natural killer (NK) cells. T cells include, but are not limited to, naïve T cells, helper T cells (CD4+), cytotoxic T cells (CD8+), regulatory T cells (Treg), central memory T cells (T_{CM}), effector memory T cells (T_{EM}), stem cell memory T cells (T_{SCM}), chimeric antigen receptor (CAR)-T cells, TCR-T cells, or any combination thereof. B cells include, but are not limited to, naïve B bells, plasma cells, memory B cells, or any combination thereof.

A cell sample may be a peripheral blood mononuclear cell sample.

Cell samples (such as a biopsy sample) may be obtained from a mammal by any suitable means of obtaining the sample using well-known and routine clinical methods. For example, procedures for drawing and processing tissue samples such as from a needle aspiration biopsy are well-known and may be employed to obtain a sample for use in the methods provided. Typically, for collection of such a tissue sample, a thin hollow needle is inserted into a mass such as a tumor mass for sampling of cells that, after being stained, will be examined under a microscope.

A cell sample may comprise cells of a tumor cells line. Illustrative tumor cell lines include, but are not limited to, cell samples from tumor cell lines such as 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLcell lysates, HC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In certain embodiments, the methods are performed on live cells, dead cells, cell lysates, nuclei, or samples enriched in cytoplasmic-, nuclear-, nucleoplasm-, euchromatin-, or heterochromatin-associated proteins. In particular embodiments, a protein is considered to be associated with a particular cellular or nuclear fraction when the majority of the protein is present within that cellular or nuclear fraction. Accordingly, the methods described herein include modified methods wherein instead of the cell being contacted with the electrophilic probe, a cell lysate, nuclei, or one or more nuclear fraction prepared from the cells are contacted with the electrophilic probe. Accordingly, certain steps outlined herein, such as lysing cells, isolating nuclei, and/or extracting nuclear fractions may not need to be performed, depending on the starting material.

### Electrophilic Probes

The reactivity of cysteine residues within cellular proteins is determined using an electrophilic probe that binds to reactive cysteine residues. In particular embodiments, the electrophilic probe preferentially or exclusively binds to cysteine amino acids and no other amino acids present in the cellular proteins. In particular embodiments, the electrophilic probe covalently binds the thiol group of cysteines that are not already bound, e.g., in a disulfide bond, as illustrated below with iodoacetamide (IAA) as the electrophilic probe binding a reactive cysteine in an enzyme.

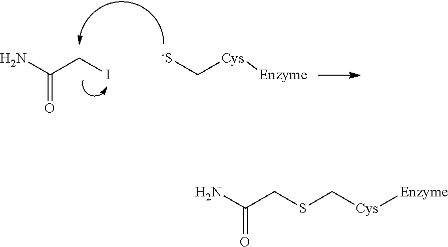

Preferably, the electrophilic probe is capable of transporting, passively or actively, across the cell membrane when contacted with intact cells, e.g., live cells. In addition, when bound, the electrophilic probe preferably alters the molecular weight of the protein, or a fragment thereof, comprising the reactive cysteine sufficiently to be identified by mass spectrometry.

In certain embodiments, the electrophilic probe comprises a reactive group selected from IAAs, chloroacetamides, epoxides, acrylamides, acyl halogens, sulfonate esters, acyloxymethyl ketones, vinylsulfonamides, propynamides, and malemides.

In certain embodiments, the electrophilic probe is an acrylamide or a derivative thereof, an IAA or a derivative thereof, a chloroacetamide or a derivative thereof, a propynamide or a derivative thereof, or a malemide or a derivative thereof. In particular embodiments, the electrophilic probe is any molecule that contains an acrylamide, iodoacetamide, chloroacetamide, propynamide, or malemide.

In certain embodiments, the electrophilic probe is an IAA, IAA derivative, or IAA-based electrophilic probe. Examples include, but are not limited to n-methyl-IAA (NM-IAA), N-(tert-butyl)-2-IAA, 4-nitrophenyl IAA, and N-[(4-hydroxyphenyl)methyl]-2-IAA.

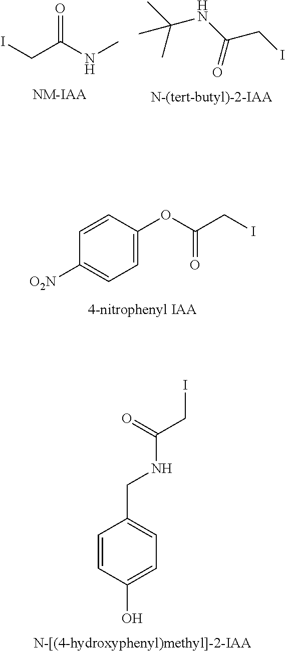

In certain embodiments, the electrophilic probe is any modified form of IAA, including but not limited to, N-isopropyliodoacetamide, N-ethyliodoacetamide, iodoacetic acid, isotopically labeled IAA, and chloroacetamide derivatives including n-methyl chloroacetamide.

In certain embodiments, the electrophilic probe is an acrylamide or acrylamide derivative.

In certain embodiments, the electrophilic probe is a chloroacetamide or chloroacetamide derivative.

In certain embodiments, the electrophilic probe is a propynamide or propynamide derivative.

In certain embodiments, the electrophilic probe is a malemide or malemide derivative.

In particular embodiments, any of the electrophilic probes, or any of the above molecules, may be derivatized to allow for pull-down assays, and include, for example: Biotin-iodoacetamide (Biotin-IAA), Iodoacetamide-alkyne (IA-alkyne) or chloroacetamide-alkyne (CA-alkyne), which allows for click-chemistry for pull-down experiments.

### Labeling Cells

The cells or cell sample (or components thereof) can be labeled by contacting the cells or cell sample with an electrophilic probe under conditions and for a time sufficient for the probe to enter the cells and bind to reactive cysteines. In certain embodiments, the cells are present in a tissue culture container, and the electrophilic probe is added to the culture media. In certain embodiments, the cells are washed and placed into a tissue culture container, e.g., wells of a culture plate, in culture media (with or without serum) prior to contacting them with the electrophilic probe. In particular embodiments, the contacting occurs at about 37° C. In certain embodiments, the cell sample is dead cells, nuclei, cell lysates, or fractions of nuclear proteins, such as nucleoplasm, euchromatin, or heterochromatin fractions.

In certain embodiments, the cells or cell sample are contacted with the electrophilic probe at a concentration of about 1 μM to about 500 μM. For example, when the electrophilic probe is NM-IAA, in certain embodiments, the cells are contacted with a concentration of NM-IAA of about 1 μM to about 200 μM, about 1 μM to about 100 μM, about 1 μM to about 50 μM, about 5 μM to about 25 μM, or about 10 μM, about 12.5 μM, or about 15 μM NM-IAA.

In particular embodiments, the cells or cell sample are contacted with the electrophilic probe, e.g., NM-IAA, for at least about 1 to at least about 24 hours, at least about 1 to about 12 hours, or at least about 1 to about 8 hours, e.g., at least or about 1, at least or about 2, at least or about 3, at least or about 4, at least or about 5, at least or about 6, at least or about 7, at least or about 8, at least or about 10, at least or about 12, at least or about 16, at least or about 20, or at least or about 24 hours.

### Cell Processing and Protein Extraction

Following treatment with the electrophilic label, the cells may be washed, harvested, and/or lysed, e.g., to extract proteins from other cellular material. In certain embodiments, e.g., wherein the cell sample is nuclei, dead cells, cell lysates, or fractions of nuclear proteins, such as ChESS fractions, this step is not performed. However, the cell sample may be washed.

Cells may be harvested by centrifugation, and washed, e.g., with phosphate-buffered saline (PBS), and then re-centrifuged.

The pelleted cells are then resuspended in a first suspension buffer. In particular embodiments, the first suspension buffer does not comprise polycations such as spermidine or spermine. The first suspension buffer may be ionic or non-ionic. In one embodiment, the first suspension buffer comprises 20 mM HEPES, 10 mM KCl, 20% glycerol, 1 mM MnCl₂, and 0.1% Triton X-100. In another embodiment, the first suspension buffer comprises 15 mM Tris pH 8.0, 15 mM NaCl, 60 mM KCl, 1 mM ethylenediaminetetraacetic acid (EDTA) pH 8.0, 0.5 mM egtazic acid (EGTA) pH 8.0, and 0.12% NP-40.

The resuspended cells are lysed by means known in the art. In certain embodiments, a detergent is added to the first suspension buffer to lyse the resuspended cells. In certain embodiments, a detergent solution is added to the resuspended cells. The detergent solution may be ionic or non-ionic. In certain embodiments, the detergent comprises NP40, SDS, Triton X-100, and/or Tween20. The detergent may be a mass-spectrometry acid-labile detergent. In particular embodiments, the detergent (e.g., NP40, Triton X-100) is added at a concentration up to 4%. In particular embodiments, the detergent is added at a concentration of from 0.01% to 4% or from 0.1% to 4%. For example, the detergent solution may be added at a concentration of from 0.01% to 0.1%. The cells are contacted with the first suspension buffer comprising a detergent for a time sufficient to lyse the cells. In certain embodiments, other methods may be used to lyse the cells, such as, e.g., sonication or physical cutting or mincing.

The nuclei of the lysed cells may then be collected via centrifugation, e.g., at about 400 g for about 5 minutes. The remaining supernatant may be collected as a cytoplasmic fraction. Any remaining unreacted cysteines present in proteins in the cytoplasmic fraction may be quenched by treatment with IAA, e.g., 10 μM IAA.

Optionally, the collected nuclei are treated with a nuclease, e.g., micrococcal nuclease (MNase). For example, the nuclei may be treated with an MNase at a temperature of about 0° C., about 10° C., about 20° C., about 30° C., about 37° C., about 40° C., or about 50° C. In certain embodiments, the nuclei may be treated with an MNase at a temperature that is within a range defined by any two of the preceding values. In certain embodiments, the nuclei may be treated with an MNase for a period of about 1 minute, about 2 minutes, about 5 minutes, about 10 minutes, about 20 minutes, about 50 minutes, or about 100 minutes. The nuclei may also be treated with an MNase for a period that is within a range defined by any two of the preceding values.

Optionally, the collected nuclei are quenched by contacting them with EDTA, EGTA, and/or one or more protease inhibitor. In certain embodiments, the nuclei are quenched with a final concentration of EDTA or EGTA of from about 0.1 mM to about 10 mM.

Following collection and optional treatment with an MNase, EDTA, EGTA, and/or protease inhibitor(s), the nuclei are resuspended in a second suspension buffer or solution. In particular embodiments, the second suspension buffer does not comprise polycations such as spermidine or spermine. In particular embodiments, the second suspension buffer comprises a salt. The second suspension solution may comprise the first suspension solution and a salt. The salt may comprise an alkali or alkaline earth salt, such as a lithium salt, a sodium salt, a potassium salt, a rubidium salt, a magnesium salt, a calcium salt, or a strontium salt. The salt may comprise a halide salt, such as a fluoride salt, a chloride salt, a bromide salt, or an iodide salt. The salt may comprise an alkali halide salt, such as lithium fluoride, lithium chloride, lithium bromide, lithium iodide, sodium fluoride, sodium chloride, sodium bromide, sodium iodide, potassium fluoride, potassium chloride, potassium bromide, or potassium iodide. The salt may comprise an alkaline earth halide salt, such as magnesium fluoride, magnesium chloride, magnesium bromide, magnesium iodide, calcium fluoride, calcium chloride, calcium bromide, calcium iodide, strontium fluoride, strontium chloride, strontium bromide, or strontium iodide. The salt may comprise an ammonium salt. The salt may comprise a transition metal salt. The salt may comprise an acetate, benzoate, carbonate, chromate, citrate, cyanide, hypochlorite, chlorite, chlorate, perchlorate, dichromate, dihydrogen phosphate, bicarbonate, bisulfate, hydrogen phosphate, hydroxide, nitrite, nitrate, peroxide, permanganate, phosphate, sulfite, or sulfate salt. In particular embodiments, the salt is sodium chloride.

In particular embodiments, the second suspension buffer comprises an isotonic buffer, a low salt buffer, and/or a high salt buffer. In particular embodiments, an isotonic buffer has approximately physiological salt concentrations matching the nuclear environment, and in certain embodiments, an isotonic buffer has a salt, e.g., NaCl, at a concentration of about 10 mM to about 20 mM, e.g., about 15 mM. In particular embodiments, a low salt buffer has a salt, e.g., NaCl, at a concentration of about 100 mM to about 400 mM or about 100 mM to about 200 mM, e.g., about 150 mM, about 160 mM, or about 250 mM. In particular embodiments, a high salt buffer has a salt, e.g., NaCl, at a concentration of about 400 mM to about 800 mM, e.g., about 600 mM. In certain embodiments, the second suspension buffer comprises a series of buffers, e.g., an isotonic buffer, a low salt buffer, and/or a high salt buffer as described above, with increasing salt concentrations for sequential separation of nuclear or chromatin proteins into different fractions.

The resuspended nuclei may be incubated in the isotonic, low salt, and/or high salt buffer. In certain embodiments, the nuclei may be incubated at a temperature of about 0° C., about 4° C., about 10° C., about 20° C., about 40° C., or about 100° C. The nuclei may be incubated at a temperature that is within a range defined by any two of the preceding values. In certain embodiments, the nuclei may be incubated for a period of about 1 minute, about 3 minutes, about 5 minutes, about 10 minutes, about 30 minutes, about 50 minutes, or about 100 minutes. The cells may be incubated for a period that is within a range defined by any two of the preceding values.

The resuspended nuclei may then be pelleted to collect insoluble chromatin from the second suspension buffer. The pelleted insoluble chromatin may be saved for further analysis. The remaining supernatant may be collected as a nuclear fraction. The supernatant liquid may be enriched in nuclear proteins. Any remaining unreacted cysteines present in proteins in the nuclear fraction may be quenched by treatment with IAA, e.g., 10 μM IAA.

Following collection and optional treatment with an MNase, RNase, DNase, Benzonase, other nucleases, EDTA, EGTA, and/or protease inhibitor(s) as described above, the nuclei may be resuspended in a sequential series of suspension solutions comprising increasing salt, e.g., NaCl, concentrations, in order to selectively extract various subsets of nuclear proteins, including nucleoplasm-enriched proteins, euchromatin-enriched proteins, and/or heterochromatin-enriched proteins. For example, (1) the nuclei may be resuspended and incubated in an isotonic buffer to extract nucleoplasm-enriched proteins, followed by centrifugation to collect supernatant as a nucleoplasm fraction and insoluble chromatin; (2) the insoluble chromatin resulting from (1) may be resuspended and incubated in a low salt buffer to extract euchromatin-associated proteins, followed by centrifugation to collect supernatant as an euchromatin fraction and insoluble chromatin; and (3) the insoluble chromatin resulting from (2) may be resuspended and incubated in a high salt buffer to extract heterochromatin-associated proteins, following by centrifugation to collect supernatant as a heterochromatin fraction. Any remaining unreacted cysteines present in proteins in the nucleoplasm fraction, euchromatin fraction, or heterochromatin fraction may be quenched by treatment with IAA, e.g., 10 μM IAA following their collection.

It is further understood that the samples do not necessarily have to be resuspended in each of the isotonic, low salt, and high salt buffers; instead; the method may be performed using one or two of these buffers, e.g., to selectively enrich for nuclear proteins associated with desired nuclear fraction(s). The nuclei and insoluble chromatin may be incubated in the various buffers for times and under conditions as described herein. For a non-limiting example, after extraction of nucleoplasm-enriched proteins, the nuclei may be resuspended and incubated in a single high salt buffer to extract chromatin proteins. In this example, the single high salt buffer combines the sequential low salt and high salt extractions into one step.

In particular embodiments, samples enriched for cytoplasmic proteins, nucleoplasm-associated proteins, euchromatin-associated proteins, and/or heterochromatin-associated proteins are obtained according to methods disclosed in U.S. Patent Application Publication No. 20200033358, which is incorporated herein by reference in its entirety.

In particular embodiments, any of the methods disclosed herein comprises one or more of: (1) using detergents (NP-40) for nuclear isolation to ensure that the proteins are not denatured during the process; (2) quenching with EDTA and/or use of protease inhibitors to prevent protein degradation during the process; (3) not using buffers with polycations, which interferes with protein analysis; and (4) sonication or removal of DNA/RNA by enzymatic processes.

### Isolation of Nuclear Proteins Using Lectin-Coated Magnetic Beads

In some embodiments, nuclear and chromatin proteins may be isolated from nuclei using lectin bound to a solid support (e.g., magnetic beads) to aid the transfer of nuclear pellets through serial wash buffers as described. The lectins can include one or more lectins that bind to LacNAc, galactose, and/or GalNAc. In some embodiments, the one or more lectins comprise Erythrina crista-galli lectin (ECL), Ricinus communis agglutinin I (RCA), Amaranthus caudatus lectin, Datura stramonium lectin, Lycopersicon esculentum lectin, and/or Maackia amurensis agglutinin I. In some embodiments, the lectin is ECL. In some embodiments, the lectin is RCA. In some embodiments, the lectin bound to a solid support is a lectin-coated magnetic bead.

In some embodiments, following suspension in or contacting with a suspension buffer for a time sufficient to extract nuclear proteins from the nuclei, the remaining nuclei are separated from the supernatant which now contains the nuclear proteins, e.g., through the use of the solid support. The solid support may be removed from the supernatant, or the supernatant may be removed from the solid support, e.g., through the use of a magnet to hold or move a magnetic bead solid support to which the nuclei and/or nuclear membranes are bound.

For example, after removal of the cytosolic fraction, the pellet containing the nuclei can be resuspended in a second suspension buffer, e.g., an isotonic buffer. The sample resuspended in the second suspension buffer is then added to and/or thoroughly mixed with the lectin-coupled solid support. For example, activated lectin-conjugated magnetic beads are added to the sample with the pellet and the EDTA-free isotonic buffer and mixed by pipetting up and down. In some embodiment, activated lectin-conjugated magnetic beads are added at a volume of 20 μl per million cells.

The samples are then contacted with the lectin-coupled solid support for a time sufficient for the cell nuclei to bind to the lectin-coupled solid support. In some embodiments, the contact period is at least 5, 10, or 20 minutes. In some embodiments, the sample is subjected to gentle shaking during the contact period. In some embodiments, the sample is placed on wet ice or kept at 4° C. during the contact period.

After the cell nuclei are bound to the solid support, the supernatant is separated from the cell nuclei bound to the solid support through centrifugation or other means known in the arts. For example, cell nuclei bound to beads can be centrifuged. In some embodiments, cell nuclei bound to lectin-conjugated magnetic beads are placed onto a magnetic rack and allowed to sit for a sufficient amount of time for the magnetic beads to pellet. The supernatant is then removed carefully and retained as the nucleoplasm fraction. The nucleoplasm fraction is enriched for nuclear proteins.

The above example of nuclear protein extraction from the isotonic buffer with the aid of lectin-coated magnetic beads is for illustration purposes only, and the same can be applied to other steps of nuclear or chromatin protein extraction as described herein.

The use of lectin-coupled solid supports for handling cell nuclei and the related methods described herein can be carried out in a high-throughput format, such as with the use of multi-well plates, as high-throughput methods are often more cost-effective and increase efficiency when a large number of samples are analyzed. For example, multiple samples coupled to lectin-conjugated beads may be placed within multiple wells of a 96-well plate to be centrifuged and washed. In some embodiments, the lectin-conjugated beads are magnetic and a magnet compatible with the multi-well plate is used to collect or bind the magnetic beads in order to pellet, move, or transfer the sample. In some embodiments, the magnet immobilizes the nuclei or nuclear membrane bound to the magnetic beads, while the supernatant is removed or while suspension buffer is added. In some embodiments, the magnet binds to the nuclei and/or nuclear membrane bound to the magnetic beads and used to move or transfer the samples into a vessel, such as a separate multi-well plate. In some embodiments, an automation instrument may be used to process samples as described herein. In some embodiments, the instrument can be an automated liquid handler. In some embodiment, the instrument can be a magnetic bead handling robot. In some embodiments, the instrument can be an automated sample purification or extraction system, such as the Thermo Scientific KingFisher Purification System. In some embodiments, one or more of the steps to isolate or extract the cell nuclei and/or fractions thereof can be performed using the automated system. In some embodiments, all the isolation and extraction steps are performed using the automated system.

In some embodiments, the high throughput method comprises multiple samples of cell nuclei that are present in a buffer within multiple wells of a sample plate and a solid support of magnetic beads, wherein the cell nuclei bound to the solid support are removed from the multiple wells using magnets. In some embodiments, the cell nuclei bound to the solid support are then placed into a second buffer within multiple wells of a sample plate, wherein the second buffer may be the same or a different buffer from the preceding buffer. In some embodiments, the steps of removing the cell nuclei bound to the solid support from the multiple wells using magnets and placing into a second buffer may be repeated one or more times. In some embodiments, the cell nuclei bound to the solid support, wherein the solid support is lectin-conjugated magnetic beads, are removed from the multiple wells using magnets and sequentially placed into and removed from a series of buffers, wherein the buffers comprise an isotonic buffer to extract nucleoplasm proteins, a low salt buffer to extract euchromatic proteins, and a high salt buffer to extract heterochromatin proteins.

Fractions containing cytoplasmic proteins, nucleoplasm-associated proteins, euchromatin-associated proteins, and/or heterochromatin-associated proteins may be further analyzed as described in the methods herein.

### Sample Analysis

After samples are obtained, e.g., one or more cytoplasmic fraction, nuclear fraction, nucleoplasm fraction, euchromatin fraction, and/or heterochromatin fraction, the samples are analyzed via mass spectrometry to determine the presence and/or absence of reactive cysteine residues in proteins present in the samples.

In certain embodiments, proteins in the sample(s) are digested using one or more proteases, e.g., trypsin, chymotrypsin, carboxypeptidase, pepsin, Lys-N endoproteinase, Lys-C endoproteinase, Glu-C endoproteinase, Asp-N endoproteinase, Arg-C endoproteinase, or any combination thereof, following by mass spectrometry analysis. Mass spectrometry data may be collected using a data-dependent acquisition (DDA), data-independent acquisition (DIA), or targeted data acquisition (TDA) strategies, e.g., selected/multiple reaction monitoring SRM/MRM) or parallel reaction monitoring (PRM), and peptides modified by the electrophilic label, e.g., NM-IAA, are identified based on the detection of the mass modification of NM-IAA (about 71.03711 Da) as a variable modification on cysteine residues using any proteomics search software.

In particular embodiments, samples may be analyzed using mass spectrometry (MS), such as tandem mass spectrometry (MS-MS), time-of-flight mass spectrometry (TOF-MS), quadrupole mass spectrometry (Q-MS), ion trap mass spectrometry (IT-MS), orbitrap mass spectrometry, or any combination thereof. The samples may be analyzed using a combination of chromatographic and mass spectrometric techniques, such as gas chromatography-mass spectrometry (GC-MS), liquid chromatography-mass spectrometry (LC-MS), high pressure liquid chromatography-mass spectrometry (HPLC-MS), nanoflow chromatography, or microflow chromatography.

### Uses

The methods disclosed herein may be used for a variety of purposes. For example, the methods may be used to identify reactive cysteine residues in cellular proteins, e.g., transcription factors, which can serve as potential drug targets.

The methods disclosed herein may be used to identify the subcellular location (e.g., cytoplasmic, nucleoplasm associated, euchromatin associated, or heterochromatin associated) of cellular proteins with reactive cysteine residues, e.g., transcription factors, the relative abundance of these proteins in each fraction, and/or any changes in the subcellular location of these proteins under a particular condition. The subcellular location of a cellular protein can often serve as an indication of the protein's function and/or active state. For a non-limiting example, transcription factors can only exert their functions in the cell nucleus. Moreover, the “open” (e.g., euchromatin) versus “closed” (e.g., heterochromatin) states of chromosomes affect transcription factor accessibility and transcriptional activity of genes, in that heterochromatin is more condensed and transcriptionally silent, whereas euchromatin is less condensed and more easily transcribed. Thus, the subnuclear association (e.g., euchromatin associated or heterochromatin associated) of transcription factors may be an indicator of their level of activity in driving gene transcription.

The methods disclosed herein may also be used to compare cytosolic and/or nuclear proteomes across two or more conditions. For instance, the methods described herein may be used to characterize cytosolic and/or nuclear proteome changes in response to one or more external or internal perturbations. Such perturbations may include, but are not limited to, a change in cell state (e.g., cell cycle), cell environment, or exposure of the cell to a chemical treatment or physical stress. Such changes may be detected by changes in the proteins having reactive cysteines present, changes in the location of reactive cysteines within proteins, and/or changes in the location of proteins, e.g., proteins moving from one or more of the cytoplasmic, nucleoplasm, euchromatin, or heterochromatin. For example, the methods may be used to identify functional systems or proteins, e.g., transcription factors, with alterations associated with an external or internal perturbations, such as alterations in the presence of reactive cysteines and or cellular location. The methods may also be used to identify functional cysteines associated with perturbations, e.g., to identify potential drug targets, by identifying cysteines with gain or loss of the ability to bind the electrophilic probe following perturbation.

In certain embodiments, the methods are used to identify cytosolic and/or nuclear proteins, e.g., transcription factors, having a change in cysteine reactivity or subcellular location following treatment with a drug candidate. In certain embodiments, the methods are used to identify cytosolic and/or nuclear proteins, e.g., transcription factors, having a change in cysteine reactivity or subcellular location in diseased cells as compared to healthy cells.

In certain embodiments, the methods may be used to diagnose a disease or disorder, including any of those described herein, including but not limited to, cancer, infection, immunological disease, metabolic disease, cardiac disease, inflammatory disease, etc. For example, a cell sample obtained from a subject may be analyzed as described herein, and results related to the cysteine reactivity and/or subcellular location of cellular proteins (e.g., transcription factors or kinases) can be compared to the results obtained from healthy cells and/or diseased cells, or a predetermined set of results from healthy cells and/or diseased cells, thereby determining whether the cells are healthy or diseased, cytosolic and/or nuclear proteins, e.g., transcription factors, having a change in cysteine reactivity or subcellular location following treatment with a drug candidate. In certain embodiments, the methods are used to identify cytosolic and/or nuclear proteins, e.g., transcription factors, having a change in cysteine reactivity or subcellular location in diseased cells as compared to healthy cells.

In particular embodiments, the methods are used to identify and/or analyze transcription factors, transcription cofactors, kinases, DNA damage proteins, and/or proteins involved in nuclear import/trafficking.

In certain embodiments, the methods disclosed herein may be used to characterize small molecule degradation compounds. The methods described herein, such as methods 100, 200, or 300, may be used to screen small molecule degraders in an unbiased manner to identify proteins targeted for degradation in response to treatment.

In certain embodiments, the methods disclosed herein may be used to assay cellular thermal shifts. The assay may detect compound engagement with the protein target in living cells by measuring changes in thermal stability of the protein. The methods described herein may be used to profile thermal stability of the nuclear proteome and to study compounds interacting with nuclear proteins.

In certain embodiments, the methods disclosed herein may be used to characterize genome edits. Genome editing with clustered regularly interspersed palindromic repeats (CRISPR)-based genome editing techniques, transcription activator-like effector nuclear (TALEN)-based genome editing techniques, zinc finger-based genome editing techniques, or other nuclease technologies can create mutations in the DNA of a cell. These mutations may result in global changes in the proteome and/or the nuclear proteome which may be detected with the methods described herein.

### Related Devices, Computer-Readable Storage Media, Computer Program, Software, and Applications

The disclosure also provides digital processing devices, computer-readable storage media, computer programs, software, web or mobile applications, standalone applications, and computer servers, including but not limited to any disclosed in

In certain embodiments, the digital processing device may include one or more hardware central processing units (CPU) that carry out the device's functions. The digital processing device may further comprise an operating system configured to perform executable instructions. In some instances, the digital processing device is connected to a computer network, is connected to the Internet such that it accesses the World Wide Web, and/or is connected to a cloud computing infrastructure. In some instances, the digital processing device is connected to an intranet. In some instances, the digital processing device is connected to a data storage device.

In accordance with the description herein, suitable digital processing device may include, by way of non-limiting examples, a server computer, a desktop computer, a laptop computer, a notebook computer, a sub-notebook computer, a netbook computer, a netpad computer, a set-top computer, a media streaming device, a handheld computer, an Internet appliance, a mobile smartphone, a tablet computer, a personal digital assistant, a video game console, and a vehicle. Those of skill in the art will recognize that many smartphones may be suitable for use in the system described herein. Those of skill in the art will also recognize that select televisions, video players, and digital music players with optional computer network connectivity may be suitable for use in the system described herein. Suitable tablet computers may include those with booklet, slate, and convertible configurations, known to those of skill in the art.

The digital processing device may include an operating system configured to perform executable instructions. The operating system may be, for example, software, including programs and data, which may manage the device's hardware and provides services for execution of applications. Those of skill in the art will recognize that suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some cases, the operating system is provided by cloud computing. Those of skill in the art will also recognize that suitable mobile smart phone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems may include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems may include, by way of non-limiting examples, Sony® PS3®, Sony® PS4®, Microsoft® Xbox 360®, Microsoft Xbox One, Nintendo® Wii®, Nintendo® Wii U®, and Ouya®.

The systems, apparatus, and methods disclosed herein may include one or more non-transitory computer readable storage media encoded with a program including instructions executable by the operating system of an optionally networked digital processing device. In further instances, a computer readable storage medium is a tangible component of a digital processing device. In still further instances, a computer readable storage medium may be removable from a digital processing device. A computer readable storage medium may include, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

The systems, apparatus, and methods disclosed herein may include at least one computer program, or use of the same. A computer program may include a sequence of instructions, executable in the digital processing device's CPU, and written to perform a specified task. In some embodiments, computer readable instructions are implemented as program modules, such as functions, objects, Application Programming Interfaces (APIs), data structures, and the like, that perform particular tasks or implement particular abstract data types. In light of the disclosure provided herein, those of skill in the art will recognize that a computer program, in certain embodiments, may be written in various versions of various languages.

The functionality of the computer readable instructions may be combined or distributed as desired in various environments. A computer program may comprise one sequence of instructions. A computer program may comprise a plurality of sequences of instructions. In some instances, a computer program is provided from one location. In other instances, a computer program is provided from a plurality of locations. In additional cases, a computer program includes one or more software modules. Sometimes, a computer program may include, in part or in whole, one or more web applications, one or more mobile applications, one or more standalone applications, one or more web browser plug-ins, extensions, add-ins, or add-ons, or combinations thereof.

The systems and methods disclosed herein may include software, server, and/or database modules, or use of the same. In view of the disclosure provided herein, software modules may be created by techniques known to those of skill in the art using machines, software, and languages known to the art. The software modules disclosed herein may be implemented in a multitude of ways. A software module may comprise a file, a section of code, a programming object, a programming structure, or combinations thereof. A software module may comprise a plurality of files, a plurality of sections of code, a plurality of programming objects, a plurality of programming structures, or combinations thereof. In various aspects, the one or more software modules comprise, by way of non-limiting examples, a web application, a mobile application, and a standalone application. In some instances, software modules are in one computer program or application. In other instances, software modules are in more than one computer program or application. In some cases, software modules are hosted on one machine. In other cases, software modules are hosted on more than one machine. Sometimes, software modules may be hosted on cloud computing platforms. Other times, software modules may be hosted on one or more machines in one location. In additional cases, software modules are hosted on one or more machines in more than one location.

The methods, apparatus, and systems disclosed herein may include one or more databases, or use of the same. In view of the disclosure provided herein, those of skill in the art will recognize that many databases may be suitable for storage and retrieval of analytical information described elsewhere herein. In various aspects described herein, suitable databases may include, by way of non-limiting examples, relational databases, non-relational databases, object oriented databases, object databases, entity-relationship model databases, associative databases, and XML databases. A database may be internet-based. A database may be web-based. A database may be cloud computing-based. Alternatively, a database may be based on one or more local computer storage devices.

Methods and systems described herein may further be performed as a service. For example, a service provider may obtain a sample that a customer wishes to analyze. The service provider may then encode the sample to be analyzed by any of the methods described herein, and may perform the analysis and provide a report to the customer. The customer may also perform the analysis and provide the results to the service provider for decoding. In some instances, the service provider then provides the decoded results to the customer. In other instances, the customer may receive encoded analysis of the samples from the provider and may decode the results by interacting with software installed locally (at the customer's location) or remotely (e.g., on a server reachable through a network). Sometimes, the software may generate a report and transmit the report to the costumer. Exemplary customers may include clinical laboratories, hospitals, industrial manufacturers, and the like. Sometimes, a customer or party may be any suitable customer or party with a need or desire to use the methods provided herein.

### EXAMPLES

### Example 1

### Identification of Reactive Cysteines in Nuclear Proteins by Chromatin Extraction with Salt Separation from Live Cells

In this example, methods using an electrophilic probe and nuclear protein enrichment were conducted to identify reactive cysteines in nuclear proteins, including transcription factors.

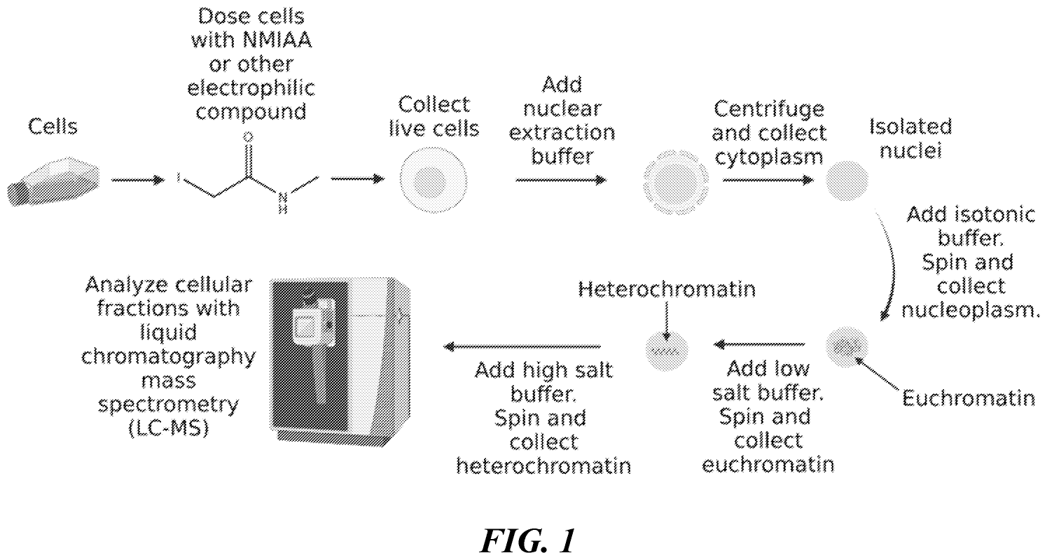

Five million cells, from leukemia, myeloma, or other cancer cell cultures, were treated with 12.5 μM n-methyl iodoacetamide (NM-IAA) in triplicate for 4 hours. The cells were then washed with phosphate-buffered saline (PBS) and fractionated using chromatin enriching salt separation (ChESS). ChESS used four sequential buffer washes of increasing ionic strength to separate the cellular proteome into cytosolic-, nucleoplasm-, euchromatin-, and heterochromatin-enriched fractions, as outlined in FIG. 1. Each of the fractions was digested using trypsin and analyzed using a data-dependent acquisition (DDA) strategy on an Evosep One-ThermoFisher Exploris 480 orbitrap platform. The results were analyzed using the Comet search tool with the mass of the NM-IAA addition as a variable modification on cysteine residues, then filtered at a 1% false discovery rate with Peptide Prophet or mokapot. DDA results were quantified with FlashLFQ. Each sample was also acquired using a data independent acquisition (DIA) approach on the same mass spectrometer in order to accurately quantify the peptides and proteins in each sample. DIA samples were analyzed using EncyclopeDIA with a spectral library that was generated using the results from DDA analysis and Comet search results or using a predicted spectral library from Prosit.

Cells were grown, treated, harvested, and nuclear fractions collected as follows.

Dose Cells with NM-IAA

THP-1 acute monocytic leukemia cells were grown to 95% viability. 1-5 million cells were collected for each sample and transferred into individual wells of a culture plate. Triplicate samples were prepared for NM-IAA treatment and for the DMSO control.

12.5 μM NM-IAA in DMSO was added to the appropriate wells, and the cells were incubated at 37° C. for 4 hours.

### Harvest and Lyse Cells

Cells for each condition were isolated by spinning in a centrifuge at 400 g for 5 min.

Freshly harvested cells were washed with ice cold PBS, and spun at 400 g for 5 min.

Cells were resuspended in 200 uL buffer A (15 mM Tris pH 8.0, 15 mM NaCl, 60 mM KCl, 1 mM EDTA pH 8.0, 0.5 mM EGTA pH 8.0, 0.12% NP-40) and mixed by pipetting up and down gently.

200 uL buffer A was added to the cell suspension to lyse the cells. Buffer A can be substituted with nuclear extraction buffer (20 mM HEPES, 10 mM KCl, 20% glycerol, 1 mM MnCl₂, 0.1% Triton X-100).

Nuclei were pelleted at 400 g for 5 minutes.

Supernatant was removed into a clean microcentrifuge tube and set aside as CYTOPLASMIC fraction.

### Fractionate Chromatin

The nuclear pellet was resuspended in 200 uL isotonic buffer (10 mM Tris pH 8.0, 15 mM NaCl, 60 mM KCl, 1.5 mM EDTA pH 8.0) and incubated at 4° C. for 15 minutes.

The nuclear pellet was centrifuged at 500 g for 5 minutes.

Supernatant was removed into a clean microcentrifuge tube and set aside as NUCLEAR fraction.

The nuclear pellet was resuspended in 200 uL euchromatin extraction buffer (10 mM Tris pH 8.0, 250 mM NaCl, 1 mM EDTA pH 8.0) and incubated at 4° C. for 15 minutes.

The nuclear pellet was centrifuged at 1000 g for 5 minutes.

Supernatant was removed into a clean microcentrifuge tube and set aside as EUCHROMATIN fraction.

The nuclear pellet was resuspended in 200 uL heterochromatin extraction buffer (10 mM Tris pH 8.0, 600 mM NaCl, 1 mM EDTA pH 8.0) and incubated at 4° C. for 15 minutes. The nuclear pellet was centrifuged at 20,000 g for 10 minutes.

Supernatant was removed into a clean microcentrifuge tube and set aside as HETEROCHROMATIN fraction.

10 μM IAA was added to each of the fractions (CYTOPLASMIC, NUCLEAR, EUCHROMATIN, and HETEROCHROMATIN) to quench any remaining unreacted cysteines before proceeding.

### Analyze Samples Using Mass Spectrometry

Proteins in the four fractions (CYTOPLASM, NUCLEAR, EUCHROMATIN, and HETEROCHROMATIN) were digested using trypsin protease according to the manufacturer's instructions.

Mass spectrometry data was collected using a DDA strategy.

Peptides that were modified with NM-IAA were identified by using the mass modification of NM-IAA (71.03711 Da) as a variable modification on cysteine residue using the Comet proteomics search software.

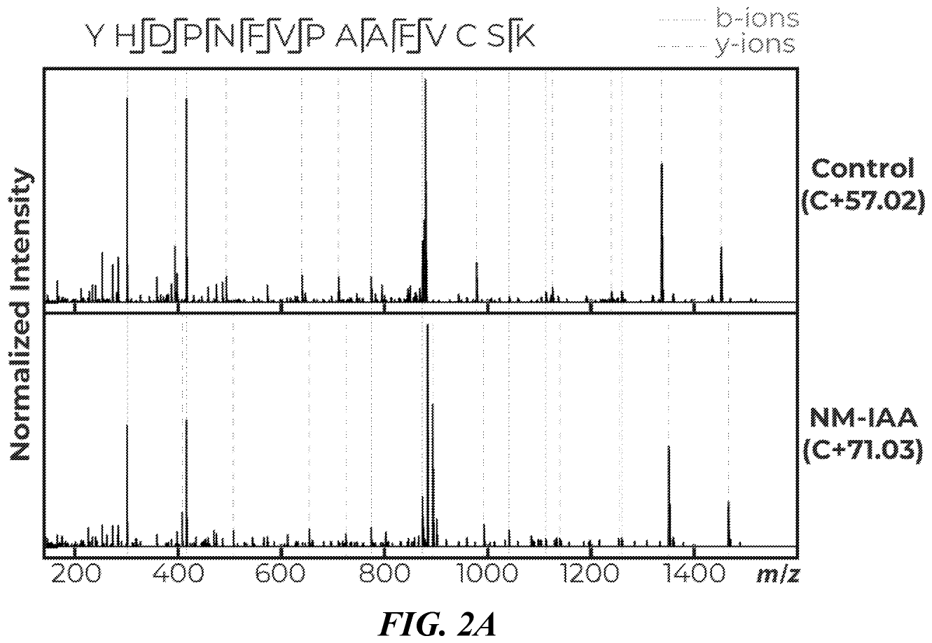

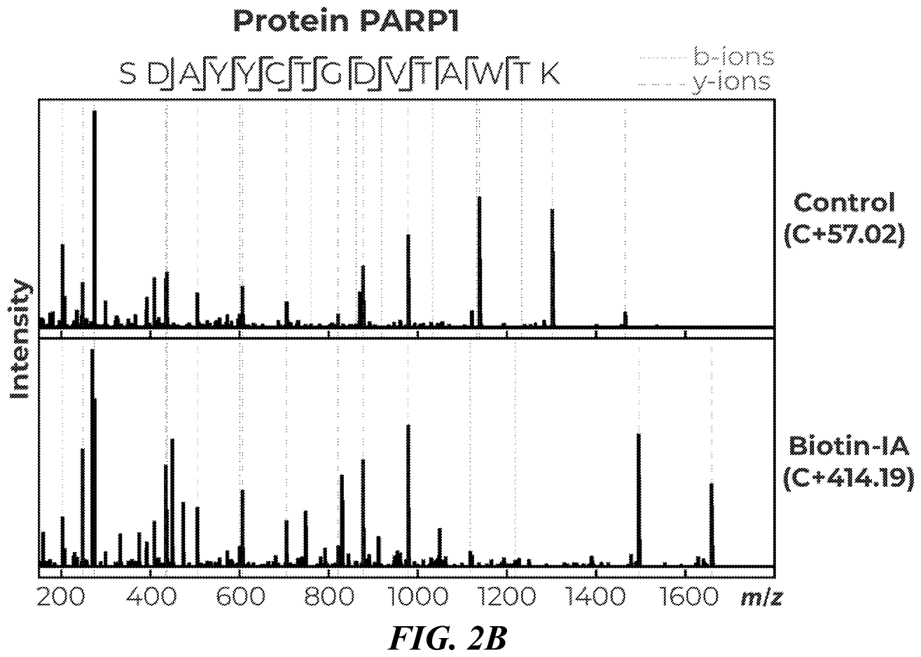

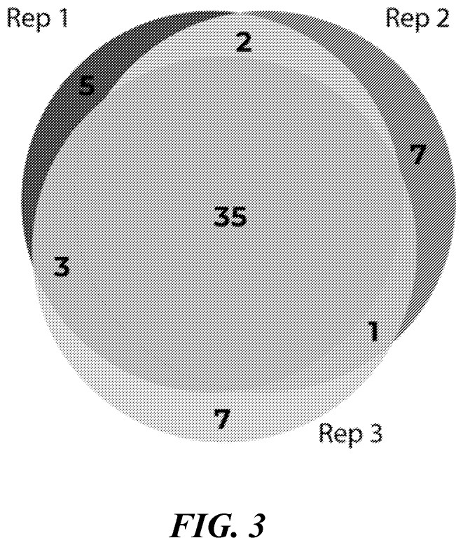

As shown in FIG. 2, reactive cysteines of transcription factor peptides that were modified with NM-IAA, e.g., a cysteine from peptide YHDPNFVPAAFVCSK (SEQ ID NO: 1) of the transcription factor CTCF in THP-1 cells, could be detected by mass spectrometry. FIG. 3 shows the number of transcription factor cysteines that were modified with NM-IAA in THP-1 cells across three replicates (Rep 1, Rep 2, and Rep 3).

Additionally, ChESS fractionation enabled the identification of dozens of NM-IAA-modified transcription factors in multiple cancer cell lines. In addition to characterizing the reactive cysteines of transcription factors, movement of a transcription factor between ChESS fraction after NM-IAA treatment indicated a functional consequence of the cysteine modification. For example, the transcription factor CTCF was ejected from heterochromatin and observed in the nucleoplasm fraction after NM-IAA modification, suggesting those cysteines influence CTCF binding to DNA.

This example demonstrates that cysteines that have been modified with NM-IAA can be readily detected in proteomics experiments using the mass of NM-IAA as a variable modification, and that NM-IAA treatment with ChESS fractionation is a powerful tool for uncovering reactive and functional cysteines of transcription factors and can be used to drive drug development.

### Example 2

### Identification of Reactive Cysteines in Nuclear Proteins by Chromatin Extraction with Salt Separation Using Single High-Salt Chromatin Extraction Buffer

In this example, methods using an electrophilic probe and nuclear protein enrichment were conducted to identify reactive cysteines in nuclear proteins, including transcription factors. Compared with Example 1, a single high salt buffer (e.g., heterochromatin extraction buffer) was used to extract the chromatin proteins.

Five million cells, from leukemia, myeloma, or ovarian cancer cell cultures, were treated with 12.5 μM NM-IAA in triplicate for 4 hours. The cells were then washed with PBS and fractionated using ChESS as outlined in Example 1.

Cells were grown, treated, harvested, and nuclear fractions collected as follows. Dose Cells with NM-IAA

THP-1 acute monocytic leukemia cells were grown to 95% viability. 1-5 million cells were collected for each sample and transferred into individual wells of a culture plate. Triplicate samples were prepared for NM-IAA treatment and for the DMSO control.

12.5 μM NM-IAA in DMSO was added to the appropriate wells, and the cells were incubated at 37° C. for 4 hours.

### Harvest and Lyse Cells

Cells for each condition were isolated by spinning in a centrifuge at 400 g for 5 min.

Freshly harvested cells were washed with ice cold PBS, and spun at 400 g for 5 min.

Cells were resuspended in 200 uL buffer A (15 mM Tris pH 8.0, 15 mM NaCl, 60 mM KCl, 1 mM EDTA pH 8.0, 0.5 mM EGTA pH 8.0, 0.12% NP-40) and mixed by pipetting up and down gently.

200 uL buffer A was added to the cell suspension to lyse the cells. Buffer A can be substituted with nuclear extraction buffer (20 mM HEPES, 10 mM KCl, 20% glycerol, 1 mM MnCl₂, 0.1% Triton X-100).

Nuclei were pelleted at 400 g for 5 minutes.

Supernatant was removed into a clean microcentrifuge tube and set aside as CYTOPLASMIC fraction.

### Fractionate Chromatin

The nuclear pellet was resuspended in 200 uL isotonic buffer (10 mM Tris pH 8.0, 15 mM NaCl, 60 mM KCl, 1.5 mM EDTA pH 8.0) and incubated at 4° C. for 15 minutes.

The nuclear pellet was centrifuged at 500 g for 5 minutes.

Supernatant was removed into a clean microcentrifuge tube and set aside as NUCLEAR fraction.

The nuclear pellet was resuspended in 200 uL heterochromatin extraction buffer (10 mM Tris pH 8.0, 600 mM NaCl, 1 mM EDTA pH 8.0) and incubated at 4° C. for 15 minutes. The nuclear pellet was centrifuged at 20,000 g for 10 minutes.

Supernatant was removed into a clean microcentrifuge tube and set aside as CHROMATIN fraction.

10 μM IAA was added to each of the fractions (CYTOPLASMIC, NUCLEAR, and CHROMATIN) to quench any remaining unreacted cysteines before proceeding with sample analysis using mass spectrometry as outlined in Example 1.

### Example 3

### Identification of Reactive Cysteines in Nuclear Proteins by Dosing Cell Nuclei with an Electrophilic Compound Before Chromatin Extraction with Salt Separation

In this example, methods using an electrophilic probe and nuclear protein enrichment were conducted to identify reactive cysteines in nuclear proteins, including transcription factors.

Five million cells from myeloma cancer cell cultures were collected, washed with PBS, and then subjected to a nuclear extraction buffer. Isolated nuclei were treated with 12.5 μM NM-IAA for 1 hour. The nuclei were fractionated using ChESS as outlined in Example 1. Cells were grown, treated, harvested, and nuclear fractions collected as follows.

### Culture, Harvest, and Lyse Cells

MM1.S acute monocytic leukemia cells were grown to 95% viability. 1-5 million cells were collected for each sample and transferred into individual wells of a culture plate. Triplicate samples were prepared for NM-IAA treatment and for the DMSO control.

Cells for each condition were isolated by spinning in a centrifuge at 400 g for 5 min.

Freshly harvested cells were washed with ice cold PBS, and spun at 400 g for 5 min.

Cells were resuspended in 200 uL buffer A (15 mM Tris pH 8.0, 15 mM NaCl, 60 mM KCl, 1 mM EDTA pH 8.0, 0.5 mM EGTA pH 8.0, 0.12% NP-40) and mixed by pipetting up and down gently.

200 uL buffer A was added to the cell suspension to lyse the cells. Buffer A can be substituted with nuclear extraction buffer (20 mM HEPES, 10 mM KCl, 20% glycerol, 1 mM MnCl₂, 0.1% Triton X-100).

Nuclei were pelleted at 400 g for 5 minutes.

Supernatant was removed into a clean microcentrifuge tube and set aside as CYTOPLASMIC fraction.

Dose Cell Nuclei with NM-IAA

Nuclei were resuspended in 200 uL buffer A.

12.5 μM NM-IAA in DMSO was added to the nuclei and incubated on wet ice for 1 hour.

### Fractionate Chromatin

The nuclear pellet was resuspended in 200 uL isotonic buffer (10 mM Tris pH 8.0, 15 mM NaCl, 60 mM KCl, 1.5 mM EDTA pH 8.0) and incubated at 4° C. for 15 minutes. The nuclear pellet was centrifuged at 500 g for 5 minutes.

Supernatant was removed into a clean microcentrifuge tube and set aside as NUCLEAR fraction.

The nuclear pellet was resuspended in 200 uL euchromatin extraction buffer (10 mM Tris pH 8.0, 250 mM NaCl, 1 mM EDTA pH 8.0) and incubated at 4° C. for 15 minutes.

The nuclear pellet was centrifuged at 1000 g for 5 minutes.

Supernatant was removed into a clean microcentrifuge tube and set aside as EUCHROMATIN fraction.

The nuclear pellet was resuspended in 200 uL heterochromatin extraction buffer (10 mM Tris pH 8.0, 600 mM NaCl, 1 mM EDTA pH 8.0) and incubated at 4° C. for 15 minutes. The nuclear pellet was centrifuged at 20,000 g for 10 minutes.

Supernatant was removed into a clean microcentrifuge tube and set aside as HETEROCHROMATIN fraction.

10 μM IAA was added to each of the fractions (CYTOPLASMIC, NUCLEAR, EUCHROMATIN, and HETEROCHROMATIN) to quench any remaining unreacted cysteines before proceeding with sample analysis using mass spectrometry as outlined in Example 1.

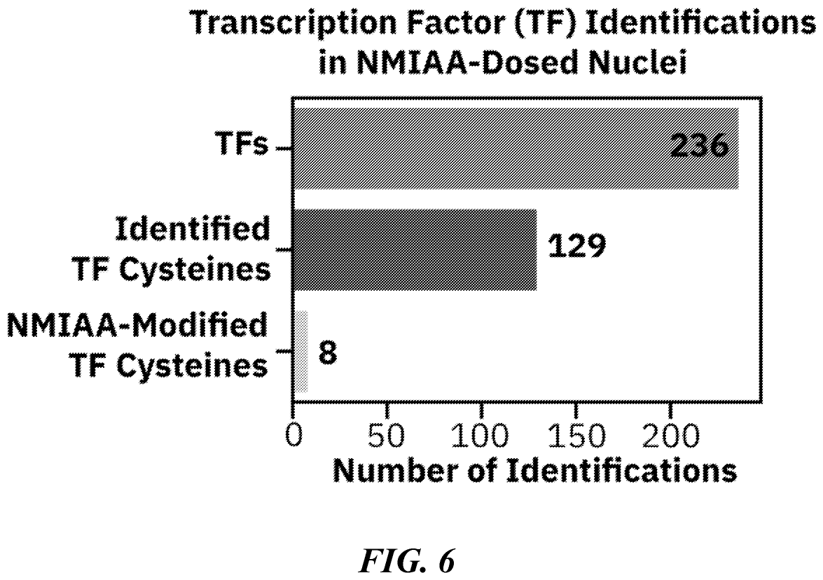

Using the methods described in the present example, MM1.S nuclei were extracted from live cells, then dosed with NM-IAA (FIG. 6), suggesting that NM-IAA can be applied directly to isolated nuclei instead of live cells. A similar number of transcription factors can be identified from compound-treated nuclei compared to live cells that are treated with a compound. Thus, treating isolated nuclei with an electrophilic compound instead of cells can be used to increase the throughput of this approach.

### Example 4

### Nuclear and Chromatin Protein Extraction Using Lectin-Coated Magnetic Beads

In this example, nuclear and chromatin proteins were extracted using lectin-coated magnetic beads, based the specific interaction of lectin and glycans enriched on the nuclear membrane. Briefly, 1-5 million cancer cells are grown to 95% viability. Cells were treated with an electrophilic covalent probe. 1-50 μM compound can be used, and treatment duration can last for any time between 0 and 72 hours or more.

Cell nuclei were isolated using nuclear extraction buffer (NEB). Nuclei are resuspended in NEB and mixed with magnetic beads that have been coated in Erythrina crista-galli lectin (ECL) or Ricinus communis agglutinin I (RCA). Table 1 below shows exemplary lectin and glycan pairs that bind with specificity.

TABLE 1
Lectin and glycan pairs that bind with specificity

| Lectin | Glycan epitope names |
|---|---|
| Amaranthus caudatus lectin | Galb1-3GalNAc, LacNAc |
| Datura Stramonium lectin | LacNAc, GlcNAcb1-4GlcNAc |
| Erythrina crista-galli lectin | LacNAc, galactose, GalNAc |
| Lycopersicon esculentum lectin | LacNAc |
| Maackia amurensis agglutinin I | 3′sulfo-LacNAc, 3′sulfo-Lac |
| Ricinus communis agglutinin I | LacNAc, galactose, GalNAc |

Lectin-coated magnetic beads were used to move the nuclei into sequential wash buffers for extraction of subsets of nuclear and chromatin proteins: (1) the nuclei were moved from NEB into an isotonic buffer to extract nucleoplasm proteins; (2) the nuclei were moved from isotonic buffer into a low-salt buffer (euchromatin buffer) to extract euchromatic proteins; and/or (3) the nuclei were moved from the euchromatin buffer into a high-salt buffer (heterochromatin buffer) to extract heterochromatin proteins. Step (2) can be omitted, as the nuclei can be moved directly from the isotonic buffer into the high salt (heterochromatin) buffer for a single chromatin extraction method. A Thermo Scientific Kingfisher robot or other magnetic bead-handling robot can be used to move the nuclei. Table 2 below summarizes the different buffers.

TABLE 2
Formulations of buffers used for isolating cell nuclei

| Buffer | Components |
|---|---|
| Bead activation buffer | 20 mM HEPES, 10 mM KCl, 10 mM |
| | CaCl₂ 1 mM MnCl₂ |
| Nuclear extraction buffer | 20 mM HEPES, 10 mM KCl, 20% |
| | Glycerol, 1 mM MnCl₂, |
| | 0.1% Triton X-100 |
| EDTA-free Isotonic buffer | 10 mM Tris pH 8.0, 15 mM NaCl, |
| | 60 mM KCl |
| Euchromatin buffer | 10 mM Tris pH 8.0, 250 mM NaCl, |
| (low salt buffer) | 1 mM EDTA pH 8.0 |
| Heterochromatin buffer | 10 mM Tris pH 8.0, 600 mM NaCl, |
| (high salt buffer) | 1 mM EDTA pH 8.0 |

Each of the fractions was digested using trypsin and analyzed as described in Example 1.

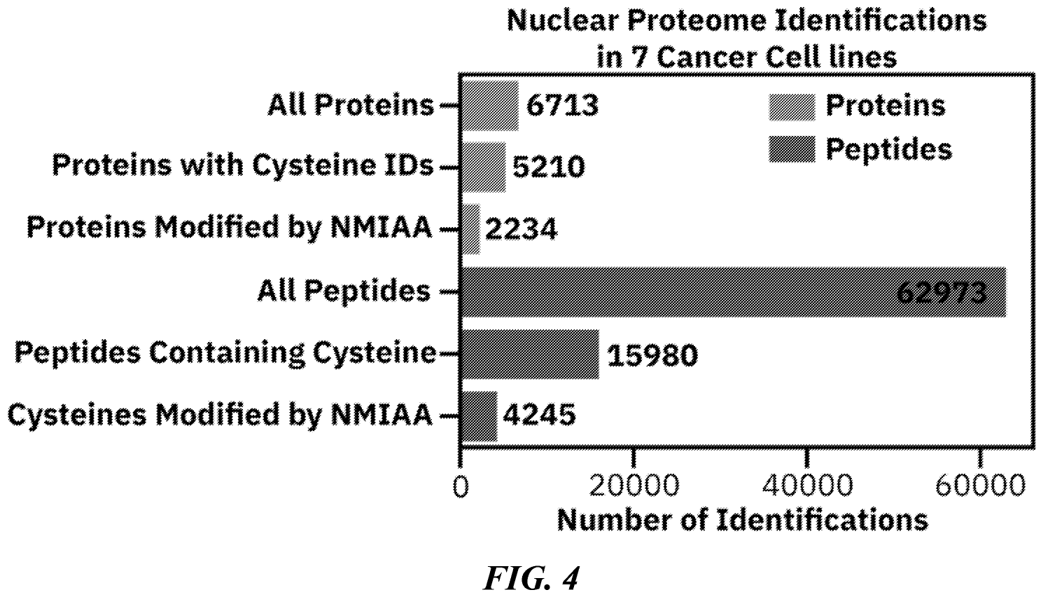

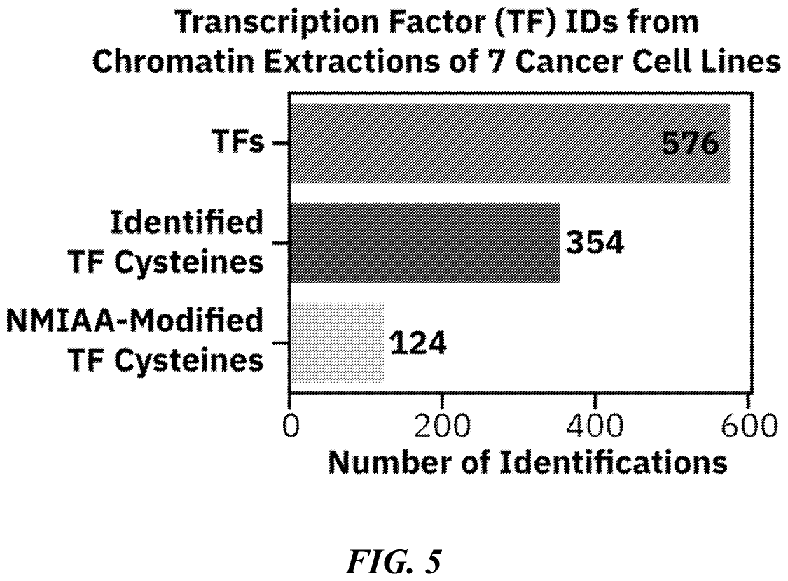

Using the methods described in Example 1 and the present example, seven cancer cell lines (THP-1, SK-N-AS, Kelly, RD, RH4, RH30, IMR32) were treated with NM-IAA. Nuclear and chromatin proteins were isolated from each sample using ChESS and analyzed by mass spectrometry. 4245 cysteines were found to be modified across the nuclear proteome (FIG. 4). Chromatin protein extraction also facilitates the identification of transcription factors. Of 576 transcription factors that were identified, a high proportion of them were found to have cysteines that react with NM-IAA (FIG. 5).

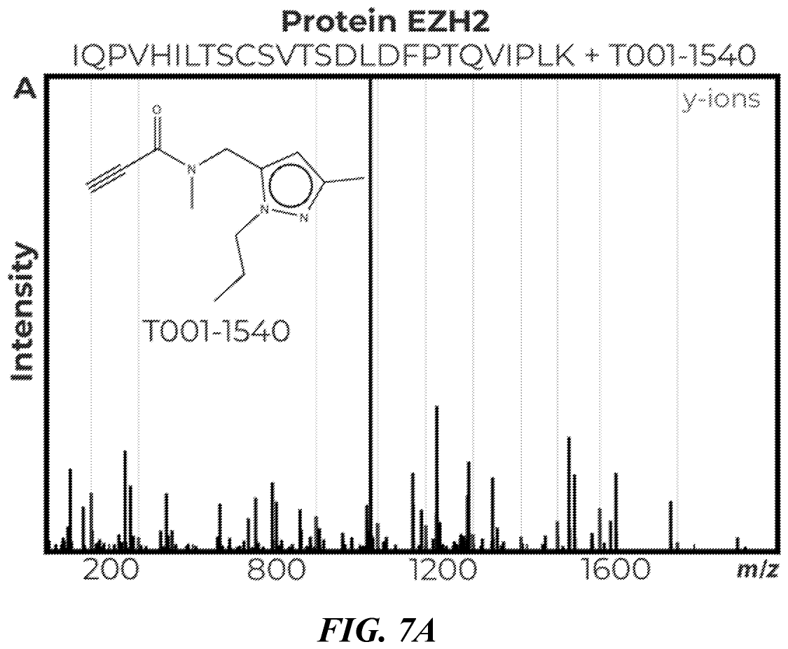

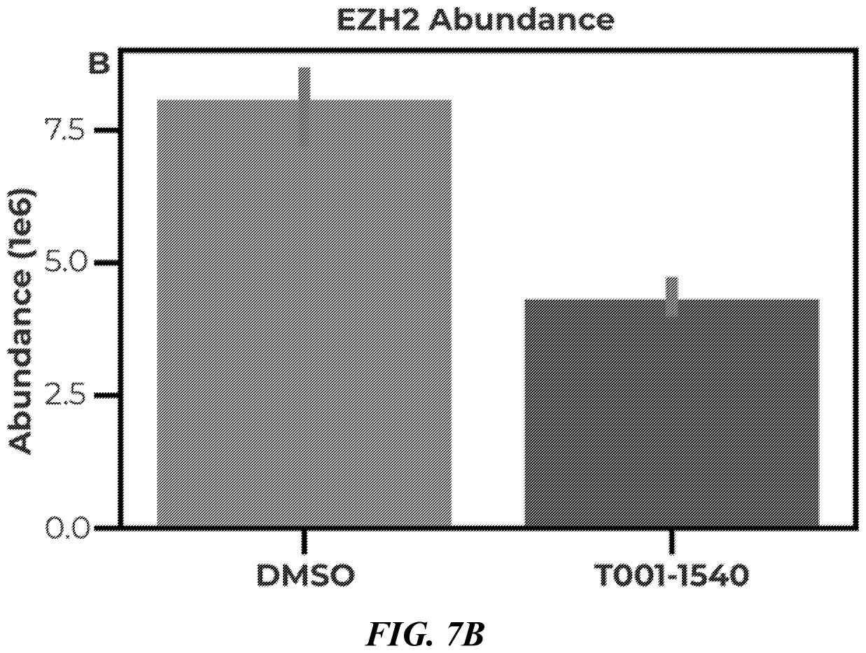

In another experiment using a single high salt (heterochromatin) buffer as described in the present example, a cysteine within a peptide of the EZH2 protein comprising a sequence of IQPVHILTSCSVTSDLDFPTQVIPLK (SEQ ID NO: 3) was found to be modified by an electrophilic molecule, T001-1540, that contains a propynamide reactive site (FIG. 7A). T001-1540 was applied to OVCAR3 cells for 2 hours in triplicate. The OVCAR3 cells were subjected to chromatin extraction, and the chromatin-associated proteins were analyzed by DIA mass spectrometry, which revealed that EZH2 was significantly depleted by T001-1540 treatment (FIG. 7B). This data indicates that treatment with an electrophilic probe can affect the abundance of chromatin-associated proteins such as EZH2, and that chromatin protein extraction can facilitate the discovery of molecules that affect the abundance or localization of chromatin-associated proteins.

### ENUMERATED EMBODIMENTS

The following enumerated embodiments provide illustrative, non-limiting examples of methods of the disclosure:

Embodiment 1. A method for identifying reactive cysteine residues present in cellular proteins, the method comprising:
(a) contacting live cells, dead cells, cell lysates, nuclei, or a nuclear fraction obtained from cells with an electrophilic probe;
(b) optionally harvesting and lysing the live cells or the dead cells of (a);
(c) optionally separating nuclei from the lysed cells of (b), and optionally collecting the supernatant from the separated nuclei as a cytoplasmic fraction;
(d) optionally extracting nuclear proteins by resuspending the nuclei of (a) or the separated nuclei of (c) in an isotonic buffer comprising salt (and optionally not comprising polycations);
(e) optionally separating insoluble chromatin and collecting the supernatant from the separated insoluble chromatin as a nuclear fraction; and
(f) performing mass spectrometry analysis of proteins present in the cytoplasmic fraction of (c) and/or the nuclear fraction of (a) or (e) to identify reactive cysteines modified by the electrophilic probe,
   thereby identifying reactive cysteines present in cellular proteins.

Embodiment 2. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) resuspending the separated nuclei of (c) in an isotonic buffer comprising salt (and optionally not comprising polycations);
(e) separating insoluble chromatin from the resuspended nuclei of (d) and collecting the supernatant from the separated insoluble chromatin as a nuclear fraction; and
(f) performing mass spectrometry analysis of proteins present in the cytoplasmic fraction and/or nuclear fraction to identify reactive cysteines modified by the electrophilic probe,
   thereby identifying reactive cysteines present in cellular proteins.

Embodiment 3. A method for identifying reactive cysteine residues present in cellular proteins, the method comprising:
(a) contacting live cells, dead cells, cell lysates, nuclei, or a nuclear fraction obtained from cells with an electrophilic probe;
(b) optionally harvesting and lysing the live cells or the dead cells of (a);
(c) optionally separating nuclei from the lysed cells of (b), and optionally collecting the supernatant from the separated nuclei as a cytoplasmic fraction, wherein the cytoplasmic fraction is enriched for cytosolic proteins;
(d) optionally extracting nucleoplasm associated proteins, euchromatin associated proteins, and/or heterochromatin associated proteins as follows:
   (i) resuspending the nuclei of (a) or the separated nuclei of (c) in an isotonic buffer comprising salt (and optionally not comprising polycations), separating insoluble chromatin, and collecting the supernatant as a nuclear fraction, wherein the nuclear fraction is enriched for nucleoplasm associated proteins;
   (ii) resuspending the insoluble chromatin of (i) in a low salt buffer comprising an increased salt concentration as compared to the isotonic buffer (and optionally not comprising polycations), separating insoluble chromatin, and collecting the supernatant as an euchromatin fraction, wherein the euchromatin fraction is enriched for euchromatin associated proteins; and/or
   (iii) resuspending the insoluble chromatin of (i) or (ii) in a high salt buffer comprising an increased salt concentration as compared to the low salt buffer (and optionally not comprising polycations), separating insoluble chromatin, and collecting the supernatant as a heterochromatin fraction, wherein the heterochromatin fraction is enriched for heterochromatin associated proteins; and
(e) performing mass spectrometry analysis of proteins present in one or more of the cytoplasmic fraction, nuclear fraction, euchromatin fraction, and heterochromatin fraction to identify reactive cysteines modified by the electrophilic probe,
   thereby identifying reactive cysteines present in cellular proteins.

Embodiment 4. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) extracting one or more nuclear protein fractions as follows:
   (i) resuspending the separated nuclei of (c) in an isotonic buffer comprising salt (and optionally not comprising polycations), separating insoluble chromatin, and collecting the supernatant as a nuclear fraction;
   (ii) resuspending the insoluble chromatin of (i) in a low salt buffer comprising an increased salt concentration as compared to the isotonic buffer (and optionally not comprising polycations), separating insoluble chromatin, and collecting the supernatant as an euchromatin fraction; and/or
   (iii) resuspending the insoluble chromatin of (i) or (ii) in a high salt buffer comprising an increased salt concentration as compared to the low salt buffer (and optionally not comprising polycations), separating insoluble chromatin, and collecting the supernatant as a heterochromatin fraction; and
(e) performing mass spectrometry analysis of proteins present in one or more of the cytoplasmic fraction, nuclear fraction, euchromatin fraction, and heterochromatin fraction to identify reactive cysteines modified by the electrophilic probe,
   thereby identifying reactive cysteines present in cellular proteins.

Embodiment 5. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) extracting one or more nuclear protein fractions as follows:
   (i) resuspending the separated nuclei of (c) in an isotonic buffer comprising salt (and optionally not comprising polycations), separating insoluble chromatin, and collecting the supernatant as a nuclear fraction; and
   (ii) resuspending the insoluble chromatin of (i) in a high salt buffer comprising an increased salt concentration as compared to the isotonic buffer (and optionally not comprising polycations), separating insoluble chromatin, and collecting the supernatant as a chromatin fraction; and
(e) performing mass spectrometry analysis of proteins present in one or more of the cytoplasmic fraction, nuclear fraction, and chromatin fraction to identify reactive cysteines modified by the electrophilic probe,
   thereby identifying reactive cysteines present in cellular proteins.

Embodiment 6. The method of any one of embodiments 1-5, further comprising homogenizing, washing, and/or pelleting the cells before lysing the cells.

Embodiment 7. The method of any one of embodiments 1-6, wherein the cells are lysed by contacting them with a first suspension buffer comprising a detergent, optionally wherein the detergent comprises NP40 or Triton X-100, and optionally wherein the detergent is present at a concentration of from about 0.1% to about 4%.

Embodiment 8. The method of any one of embodiments 1-7, further comprising incubating the nuclei and/or insoluble chromatin in one or more buffer(s) before separating the nuclei or insoluble chromatin from the supernatant.

Embodiment 9. The method of embodiment 8, wherein the nuclei are incubated at a temperature of about 4° C.

Embodiment 10. The method of embodiment 8, wherein the nuclei are incubated for a period of about 30 minutes.

Embodiment 11. The method of any one of embodiments 1-10, wherein the salt comprises sodium chloride (NaCl).

Embodiment 12. The method of embodiment 11, wherein the isotonic buffer comprises NaCl at a concentration of about at a concentration of about 10 mM to about 20 mM, the low salt buffer comprises NaCl at a concentration of about 100 mM to about 400 mM, and/or the high salt buffer comprises NaCl at a concentration of about 400 mM to about 800 mM.

Embodiment 13. The method of embodiment 12, wherein the isotonic buffer comprises NaCl at a concentration of about 15 mM, the low salt buffer comprises NaCl at a concentration of about 250 mM, and/or the high salt buffer comprises NaCl at a concentration of about 600 mM.

Embodiment 14. The method of any one of embodiments 1-13, further comprising quenching the nuclei, optionally after step (c).

Embodiment 15. The method of embodiment 14, wherein the nuclei are quenched with ethylenediaminetetraacetic acid (EDTA).

Embodiment 16. The method of embodiment 15, wherein the EDTA is present at a concentration of from 0.1 mM to 10 mM.

Embodiment 17. The method of any one of embodiments 1-16, wherein the method further comprises treating the nuclei with a nuclease.

Embodiment 18. The method of embodiment 17, wherein the nuclei are treated with the nuclease at a temperature of about 37° C. and/or for a period of about 5 minutes.

Embodiment 19. The method of any one of embodiments 1-18, further comprising adding a surfactant to the one or more supernatants and/or the insoluble chromatin.

Embodiment 20. A method for determining subcellular location of one or more cellular proteins, comprising:
(a) performing the method of any one of embodiments 1-19; and
(b) determining the subcellular location of the one or more cellular proteins,
   wherein the subcellular location is selected from the group consisting of cytoplasmic, nuclear, nucleoplasm associated, euchromatin associated, and heterochromatin associated.

Embodiment 21. A method for determining if a condition alters subcellular location of one or more cellular proteins, comprising:
(a) performing the method of any one of embodiments 1-19 on cells subjected to a first condition; and
(b) performing the method of any one of embodiments 1-19 on cells subjected to a second condition; and
(c) determining the subcellular location of the one or more cellular protein following step (a) and step (b), wherein the subcellular location is selected from the group consisting of cytoplasmic, nucleoplasm associated, euchromatin associated, and heterochromatin associated,
   thereby determining whether the first or second condition alters the subcellular location of the one or more cellular proteins.

Embodiment 22. The method of embodiment 21, wherein the first and second conditions are selected from:
(a) a first and second environmental condition;
(b) a first and second cell state;
(c) before and after treatment with one or more candidate therapeutic agent;
(d) before and after treatment with a small molecule degradation compound;
(e) before and after a gene modification of the cells, optionally by genome editing;
(f) before and after expression of a transgene in the cells;
(g) a first and second thermal condition; and
(h) healthy cells versus diseased or injured cells.

Embodiment 23. The method of any one of embodiments 20-22, wherein the cellular proteins are transcription factors.

Embodiment 24. The method of any one of embodiments 1-23, wherein the electrophilic probe comprises a reactive group selected from the group consisting of iodoacetamides, chloroacetamides, epoxides, acrylamides, acyl halogens, sulfonate esters, acyloxymethyl ketones, vinylsulfonamides, propynamides, and malemides

Embodiment 25. The method of any one of embodiments 1-24, wherein the electrophilic probe is an acrylamide or a derivative thereof, an iodoacetamide or a derivative thereof, a chloroacetamide or a derivative thereof, a propynamide or a derivative thereof, or a malemide or a derivative thereof.

Embodiment 26. The method of embodiment 25, wherein the electrophilic probe is n-methyl iodoacetamide (NM-IAA).

Embodiment 27. The method of any one of embodiments 1-26, wherein the cells comprise cancer cells.

Embodiment 28. The method of any one of embodiments 1-26, wherein the cells comprise immune cells.

### CONCLUSION

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby

All publications disclosed herein are incorporated by reference in their entirety.

I/We claim:

1. A method for identifying reactive cysteine residues present in cellular proteins, the method comprising:
(a) contacting live cells, dead cells, cell lysates, nuclei, or a nuclear fraction obtained from cells with an electrophilic probe;
(b) optionally harvesting and lysing the live cells or the dead cells of (a);
(c) optionally separating nuclei from the lysed cells of (b), and optionally collecting the supernatant from the separated nuclei as a cytoplasmic fraction;
(d) optionally extracting nuclear proteins by resuspending the nuclei of (a) or the separated nuclei of (c) in an isotonic buffer comprising salt (and optionally not comprising polycations);
(e) optionally separating insoluble chromatin and collecting the supernatant from the separated insoluble chromatin as a nuclear fraction; and
(f) performing mass spectrometry analysis of proteins present in the cytoplasmic fraction of (c) and/or the nuclear fraction of (a) or (e) to identify reactive cysteines modified by the electrophilic probe,
thereby identifying reactive cysteines present in cellular proteins.

2. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) resuspending the separated nuclei of (c) in an isotonic buffer comprising salt (and optionally not comprising polycations);
(e) separating insoluble chromatin from the resuspended nuclei of (d) and collecting the supernatant from the separated insoluble chromatin as a nuclear fraction; and
(f) performing mass spectrometry analysis of proteins present in the cytoplasmic fraction and/or nuclear fraction to identify reactive cysteines modified by the electrophilic probe,
thereby identifying reactive cysteines present in cellular proteins.

3. A method for identifying reactive cysteine residues present in cellular proteins, the method comprising:
(a) contacting live cells, dead cells, cell lysates, nuclei, or a nuclear fraction obtained from cells with an electrophilic probe;
(b) optionally harvesting and lysing the live cells or the dead cells of (a);
(c) optionally separating nuclei from the lysed cells of (b), and optionally collecting the supernatant from the separated nuclei as a cytoplasmic fraction, wherein the cytoplasmic fraction is enriched for cytosolic proteins;
(d) optionally extracting nucleoplasm associated proteins, euchromatin associated proteins, and/or heterochromatin associated proteins as follows:
(i) resuspending the nuclei of (a) or the separated nuclei of (c) in an isotonic buffer comprising salt (and optionally not comprising polycations), separating insoluble chromatin, and collecting the supernatant as a nuclear fraction, wherein the nuclear fraction is enriched for nucleoplasm associated proteins;
(ii) resuspending the insoluble chromatin of (i) in a low salt buffer comprising an increased salt concentration as compared to the isotonic buffer (and optionally not comprising polycations), separating insoluble chromatin, and collecting the supernatant as an euchromatin fraction, wherein the euchromatin fraction is enriched for euchromatin associated proteins; and/or
(iii) resuspending the insoluble chromatin of (i) or (ii) in a high salt buffer comprising an increased salt concentration as compared to the low salt buffer (and optionally not comprising polycations), separating insoluble chromatin, and collecting the supernatant as a heterochromatin fraction, wherein the heterochromatin fraction is enriched for heterochromatin associated proteins; and
(e) performing mass spectrometry analysis of proteins present in one or more of the cytoplasmic fraction, nuclear fraction, euchromatin fraction, and heterochromatin fraction to identify reactive cysteines modified by the electrophilic probe,
thereby identifying reactive cysteines present in cellular proteins.

4. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) extracting one or more nuclear protein fractions as follows:
(i) resuspending the separated nuclei of (c) in an isotonic buffer comprising salt (and optionally not comprising polycations), separating insoluble chromatin, and collecting the supernatant as a nuclear fraction;
(ii) resuspending the insoluble chromatin of (i) in a low salt buffer comprising an increased salt concentration as compared to the isotonic buffer (and optionally not comprising polycations), separating insoluble chromatin, and collecting the supernatant as an euchromatin fraction; and/or
(iii) resuspending the insoluble chromatin of (i) or (ii) in a high salt buffer comprising an increased salt concentration as compared to the low salt buffer (and optionally not comprising polycations), separating insoluble chromatin, and collecting the supernatant as a heterochromatin fraction; and
(e) performing mass spectrometry analysis of proteins present in one or more of the cytoplasmic fraction, nuclear fraction, euchromatin fraction, and heterochromatin fraction to identify reactive cysteines modified by the electrophilic probe,
thereby identifying reactive cysteines present in cellular proteins.

5. A method for identifying reactive cysteine residues present in cellular proteins, the method comprising:
(a) contacting live cells with an electrophilic probe;
(b) harvesting and lysing the cells of (a);
(c) separating nuclei from the lysed cells of (b) and collecting the supernatant from the separated nuclei as a cytoplasmic fraction;
(d) extracting one or more nuclear protein fractions as follows:
(i) resuspending the separated nuclei of (c) in an isotonic buffer comprising salt (and optionally not comprising polycations), separating insoluble chromatin, and collecting the supernatant as a nuclear fraction; and
(ii) resuspending the insoluble chromatin of (i) in a high salt buffer comprising an increased salt concentration as compared to the isotonic buffer (and optionally not comprising polycations), separating insoluble chromatin, and collecting the supernatant as a chromatin fraction; and
(e) performing mass spectrometry analysis of proteins present in one or more of the cytoplasmic fraction, nuclear fraction, and chromatin fraction to identify reactive cysteines modified by the electrophilic probe,
thereby identifying reactive cysteines present in cellular proteins.

6. The method of any one of claims 1-5, further comprising homogenizing, washing, and/or pelleting the cells before lysing the cells.

7. The method of any one of claims 1-6, wherein the cells are lysed by contacting them with a first suspension buffer comprising a detergent, optionally wherein the detergent comprises NP40 or Triton X-100, and optionally wherein the detergent is present at a concentration of from about 0.1% to about 4%.

8. The method of any one of claims 1-7, further comprising incubating the nuclei and/or insoluble chromatin in one or more buffer(s) before separating the nuclei or insoluble chromatin from the supernatant.

9. The method of claim 8, wherein the nuclei are incubated at a temperature of about 4° C.

10. The method of claim 8, wherein the nuclei are incubated for a period of about 30 minutes.

11. The method of any one of claims 1-10, wherein the salt comprises sodium chloride (NaCl).

12. The method of claim 11, wherein the isotonic buffer comprises NaCl at a concentration of about at a concentration of about 10 mM to about 20 mM, the low salt buffer comprises NaCl at a concentration of about 100 mM to about 400 mM, and/or the high salt buffer comprises NaCl at a concentration of about 400 mM to about 800 mM.

13. The method of claim 12, wherein the isotonic buffer comprises NaCl at a concentration of about 15 mM, the low salt buffer comprises NaCl at a concentration of about 250 mM, and/or the high salt buffer comprises NaCl at a concentration of about 600 mM.

14. The method of any one of claims 1-13, further comprising quenching the nuclei, optionally after step (c).

15. The method of claim 14, wherein the nuclei are quenched with ethylenediaminetetraacetic acid (EDTA).

16. The method of claim 15, wherein the EDTA is present at a concentration of from 0.1 mM to 10 mM.

17. The method of any one of claims 1-16, wherein the method further comprises treating the nuclei with a nuclease.

18. The method of claim 17, wherein the nuclei are treated with the nuclease at a temperature of about 37° C. and/or for a period of about 5 minutes.

19. The method of any one of claims 1-18, further comprising adding a surfactant to the one or more supernatants and/or the insoluble chromatin.

20. A method for determining subcellular location of one or more cellular proteins, comprising:
(a) performing the method of any one of claims 1-19; and
(b) determining the subcellular location of the one or more cellular proteins,
wherein the subcellular location is selected from the group consisting of cytoplasmic, nuclear, nucleoplasm associated, euchromatin associated, and heterochromatin associated.

21. A method for determining if a condition alters subcellular location of one or more cellular proteins, comprising:
(a) performing the method of any one of claims 1-19 on cells subjected to a first condition; and
(b) performing the method of any one of claims 1-19 on cells subjected to a second condition; and
(c) determining the subcellular location of the one or more cellular protein following step (a) and step (b), wherein the subcellular location is selected from the group consisting of cytoplasmic, nucleoplasm associated, euchromatin associated, and heterochromatin associated,
thereby determining whether the first or second condition alters the subcellular location of the one or more cellular proteins.

22. The method of claim 21, wherein the first and second conditions are selected from:
(a) a first and second environmental condition;
(b) a first and second cell state;
(c) before and after treatment with one or more candidate therapeutic agent;
(d) before and after treatment with a small molecule degradation compound;
(e) before and after a gene modification of the cells, optionally by genome editing;
(f) before and after expression of a transgene in the cells;
(g) a first and second thermal condition; and
(h) healthy cells versus diseased or injured cells.

23. The method of any one of claims 20-22, wherein the cellular proteins are transcription factors.

24. The method of any one of claims 1-23, wherein the electrophilic probe comprises a reactive group selected from the group consisting of iodoacetamides, chloroacetamides, epoxides, acrylamides, acyl halogens, sulfonate esters, acyloxymethyl ketones, vinylsulfonamides, propynamides, and malemides

25. The method of any one of claims 1-24, wherein the electrophilic probe is an acrylamide or a derivative thereof, an iodoacetamide or a derivative thereof, a chloroacetamide or a derivative thereof, a propynamide or a derivative thereof, or a malemide or a derivative thereof.

26. The method of claim 25, wherein the electrophilic probe is n-methyl iodoacetamide (NM-IAA).

27. The method of any one of claims 1-26, wherein the cells comprise cancer cells.

28. The method of any one of claims 1-26, wherein the cells comprise immune cells.


SEQUENCE LISTING (3 sequences)

SEQ ID NO: 1 - protein, 15 aa, Homo sapiens
YHDPNFVPAAFVCSK

SEQ ID NO: 2 - protein, 15 aa, Homo sapiens
SDAYYCTGDVTAWTK

SEQ ID NO: 3 - protein, 26 aa, Homo sapiens
IQPVHILTSCSVTSDLDFPTQVIPLK